(19) United States
(12) Patent Application Publication
(10) Pub. No.: US 2025/0250321 A1
(43) Pub. Date: Aug. 7, 2025

(54) Multifunctional Multispecific Multimeric Biomolecule Polymer Having Prolonged In-Vivo Duration

(71) Applicant: Onegene Biotechnology Inc., Gyeonggi-do (KR)

(72) Inventors: Sungjin Park, Gyeonggi-do (KR); Daeseong Im, Gyeonggi-do (KR); Ryuryun Kim, Gyeonggi-do (KR); Minsum Kim, Gyeonggi-do (KR); Jaeyoung Choi, Gyeonggi-do (KR)

(73) Assignee: Onegene Biotechnology Inc., Gyeonggi-do (KR)

(21) Appl. No.: 17/780,565

(22) PCT Filed: Nov. 27, 2020
(86) PCT No.: PCT/KR2020/017029

§ 371 Date: May 27, 2022

(30) Foreign Application Priority Data
Nov. 27, 2019 (KR) 10-2019-0154945

(51) Int. Cl. C07K 14/715; A61K 38/00; C07K 14/61; C07K 14/765; C12N 9/16; C12N 9/88; C12N 9/90

(52) U.S. Cl. CPC C07K 14/7155; C07K 14/61; C07K 14/765; C12N 9/16; C12N 9/88; C12N 9/90; C12Y 301/03011; C12Y 401/02013; C12Y 503/01001; A61K 38/00; C07K 2319/30; C07K 2319/95

(57) ABSTRACT

The present invention provides a multifunctional multispecific multimeric biomolecule polymer which is formed by obtaining a biomolecule, to which a ubiquitin C-terminal tag is bound, by recombinantly expressing the biomolecule from a host cell, and polyubiquitinating, in vitro, the biomolecule along with a substrate, and proteins E1 (activation enzyme), E2 (conjugation enzyme) and E3 (ligase) which are involved in ubiquitination, and thus having the biomolecule bind to a polyubiquitin scaffold which is formed by covalently bonding two or more ubiquitins. The biomolecule of the present invention may be one or more selected from the group consisting of a protein, peptide, polypeptide, antibody, antibody fragment, DNA and RNA, and, for example, by using heterologous proteins, modularized functionality may be imparted to the multifunctional multispecific biomolecule polymer. In addition, according to the present invention, the multifunctional multispecific multimeric biomolecule polymer is provided in a form that is bound to a molecule capable of increasing the in vivo duration, and thus may be used for producing drugs requiring the increased in vivo duration of efficacy.

### CROSS-REFERENCE TO RELATED APPLICATIONS

This application is the National Stage entry of PCT/KR2020/017029, filed on Nov. 27, 2020, which claims priority to Korean Application Serial No. 10-2019-0154945, filed Nov. 27, 2019, the entire disclosures of which are hereby incorporated by reference herein.

### SEQUENCE LISTING

The instant application contains a Sequence Listing which has been submitted via Patent Center and is hereby incorporated by reference in its entirety. Said ASCII copy, created on Nov. 7, 2023, is named OGB20P-0008-WO-US_ST25.txt, and is 116,944 bytes in size.

### TECHNICAL FIELD

The present invention relates to a method for preparing a biomolecule including a protein into a polymer in a multimeric form. Specifically, the present invention relates to a method for preparing a biomolecule recombinantly expressed from a host cell into a multifunctional multispecific biomolecule polymer having an increased in vivo duration using a ubiquitination system.

### BACKGROUND ART

Preparing biomolecules and/or small molecule chemical compounds including proteins, peptides, polypeptides, antibodies, DNA and RNA in a multimeric form has various advantages. For example, the physicochemical properties of protein such as solubility, gelation, thermal stability and pH stability can be improved by linking two or more homogeneous or heterologous proteins using a fusion or a cross linker (or a cross-linking agent). For example, CLEA (cross-linked enzyme aggregate), laccase formed by multiple linking through a cross linker showed more enhanced stability and performance during starch oxidation, and CLEA of another enzyme, nitrile hydratase showed an excellent increase in activity in the conversion of acrylonitrile to acrylamide, and did not lose activity during 36 recycles.

In addition, many proteins form complexes in cells to perform complex functions, which are known to be due to the proximity effect of proteins. For example, the cellulase (Novozymes Cellic® CTec3), which is produced by preparing enzymes necessary for lignocellulose degradation, such as, cellulase, beta glucosidase (β-glucosidase), hemicellulase and the like in the form of a complex mixture using a scaffold, is known to exhibit a 3.5-fold or more increased effect in the degradation of lignocellulose. In addition, such a protein in a multimeric form exhibits a channeling effect. That is, if enzymes involved in a coupled reaction are present adjacent to each other, the transfer of the intermediate is efficient and the efficiency of the entire reaction is greatly increased. In addition, it is proposed to be desirable for an increase in its efficiency to use a homogeneous or heterologous protein in a multimeric form when analyzing any substance using a protein immobilized on a bead or a substrate, or separating and/or purifying a substance to be detected.

As described above, although the protein in a multimeric form provides various advantages in industrial and medical applications, it has been known that it is difficult to fabricate a protein having such a structure. For example, there is a method of developing and producing a multimeric protein as a new fusion enzyme by designing in-frame at the genetic stage. However, since a new protein must be designed and produced, it takes a long time to develop it and it is difficult to fuse two or more enzymes in reality. In addition, in the case of a method of fabricating a protein multimer construct (CLEA) using a chemical cross linker, the activity may be inhibited because a chemical bond does not occur at a specific site but can occur anywhere on the protein surface. Proteins that form a multimer construct must be capable of being prepared through synthesis or microbial expression, and the active sites of these proteins must not be disturbed.

A method of using ubiquitin has been proposed as a method for separating and/or purifying a protein of interest. It is the method in which first, a gene encoding a protein bound to ubiquitin is expressed in prokaryotic cells to prepare a fusion protein linked to ubiquitin, and then treated with ubiquitin cleaving enzyme to effectively separate and purify only the protein of interest from the ubiquitin fusion protein. U.S. patent application Ser. No. 10/504,785 relates to the expression of a recombinant gene and the purification of the expressed protein, and it describes that the fusion protein is prepared in which the nucleotide encoding the C-terminal domain of the ubiquitin-like protein (Ubl) is operatively bound to the nucleotide encoding the protein of interest, and it is expressed in a host cell. Korean Patent Application No. 10-2005-0050824 describes the use of ubiquitin as a fusion partner in expressing a recombinant protein. In addition, Korean Patent Application No. 10-2015-0120852 relates to the use of an ubiquitin column for purifying a protein, and describes that a polyubiquitin chain is loaded on the column, and the protein is purified using in vitro ubiquitination including E2. In addition, U.S. patent application Ser. No. 12/249,334 is to solve the problem of water solubility and folding, which is a problem in preparing by expressing a recombinant protein, and describes the use of SUMO having a cleavage site recognized by Ulp1 protease (Ubl-specific protease 1) for facilitating expression, separation and purification of the recombinant protein, and for increasing the activity of the protein. However, these methods only describe the use of ubiquitin for protein expression, and do not describe or suggest the production of a protein in a multimeric form, and since the protein to be separated and purified randomly binds to ubiquitin, these methods still have a limit to separation or analysis efficiency.

On the other hand, biomolecules such as proteins or peptides or recombinantly produced proteins or peptides are unstable molecules that exhibit a short serum half-life. In particular, these proteins or peptides are very unstable when prepared in aqueous solutions for diagnostic or therapeutic purposes. In addition, such protein or peptide drugs are disadvantageous because of their short serum half-life in vivo and must be administered at a high frequency or at a higher dose. However, frequent administration of the drug causes various side effects and causes discomfort to the patient. For example, there are many known problems that occur in patients requiring frequent administration of drugs, for example, diabetic patients or patients suffering from multiple sclerosis. Various methods for increasing the in vivo stability or half-life of such biomolecules have been studied. As an example, a component capable of increasing the half-life is covalently attached to a biomolecule such as a protein or peptide. For example, it is well known that attaching polymers such as polyethylene glycol or PEG to polypeptides can increase the serum half-life of these peptides.

Accordingly, the present inventors have made ceaseless efforts to develop a method for preparing a multifunctional multispecific biomolecule polymer having a high degree of integration and an increased in vivo duration or half-life without inhibiting the activity of the protein. As a result, a biomolecule bound to ubiquitin was recombinantly expressed from a host cell and was reacted in vitro with an enzyme related to ubiquitination to form a multifunctional multimeric biomolecule polymer bound to a polyubiquitin scaffold. Based on the above, the present inventors completed the present invention.

### PRIOR ART DOCUMENT

### Patent Document

(Patent Document 1) Korean Patent Application No. 10-2005-0050824
(Patent Document 2) Korean Patent Application No. 10-2015-0120852
(Patent Document 3) U.S. patent application Ser. No. 12/249,334

### DETAILED DESCRIPTION OF THE INVENTION

### Technical Problem

As described above, an object of the present invention is to provide a multifunctional multispecific biomolecule having an increased in vivo duration by binding a target biomolecule to a polyubiquitin scaffold.

Another object of the present invention is to provide a method for preparing a multifunctional multispecific biomolecule having an increased in vivo duration by binding a target biomolecule to a polyubiquitin scaffold.

Another object of the present invention is to provide a pharmaceutical composition comprising the multifunctional multispecific biomolecule.

Another object of the present invention is to provide a method for preparing a pharmaceutical composition comprising the multifunctional multispecific biomolecule.

### Solution to Problem

In order to achieve the above objects, the present invention provides a multifunctional multispecific multimeric biomolecule polymer that is composed of a polyubiquitin scaffold which is formed by covalently bonding two or more ubiquitins, and 2 to 10 biomolecules comprising binding moieties, each specific for different binding sites, wherein the biomolecule comprises active sites that specifically bind to other biomolecules, small molecule chemical compounds or nanoparticles, and is directly bound to the N-terminus, the C-terminus, or both the N-terminus and the C-terminus of the ubiquitin or is bound by a linker; and a carrier that prolongs the in vivo stability and/or duration of the biomolecule is directly bound to the N-terminus or the C-terminus of the ubiquitin or is bound by a linker.

In one embodiment related thereto, the linker may be a combination of 1 to 30 repeats of GGGGS (SEQ ID NO: 30) or EAAAK (SEQ ID NO:31), but is not limited thereto. In another embodiment related thereto, the biomolecule bound to the N-terminus of the ubiquitin may be the distal end of the multimeric biomolecule polymer, and the biomolecule bound to the C-terminus, the N-terminus, or both the C-terminus and the N-terminus of the ubiquitin may be the proximal end of the multimeric biomolecule polymer.

As used herein, “polymer” refers to a group of monomers of a series of biomolecules linked together. The polymer may be linear or branched (branch form). When the polymer is branched, each polymer chain may be referred to as a “polymer arm.” The end of the polymer arm linked to the initiator moiety is the proximal end, and the growing-chain end of the polymer arm is the distal end.

As used herein, “linker” refers to a chemical moiety that connects two groups together. The linker may be degradable or non-degradable. The degradable linker may be a hydrolysable, enzymatically degradable, pH sensitive, photolabile, or disulfide linker, among others. Other linkers include homobifunctional and heterobifunctional linkers.

In one embodiment of the present invention, the carrier has the function of increasing the in vivo duration of the biomolecule. The carrier may be one or more selected from the group consisting of albumin, antibody fragment, Fc domain, transferrin, XTEN (genetic fusion of non-exact repeat peptide sequence), CTP (carboxy-terminal peptide), PAS (proline-alanine-serine polymer), ELK (elastin-like peptide), HAP (homo-amino acid polymer), GLK (gelatin-like protein), PEG (polyethylene glycol), and fatty acid, but is not limited thereto.

In another embodiment of the present invention, the polyubiquitin scaffold may be formed by covalently bonding a donor ubiquitin in which one or more lysines of the ubiquitin are substituted with other amino acids including arginine or alanine, and an acceptor ubiquitin in which the 6th, 11th, 27th, 29th, 33rd, 48th, or 63rd lysine from the N-terminus is substituted with other amino acids including arginine or alanine. In addition, in another embodiment of the present invention, the 73rd leucine from the N-terminus of the ubiquitin may be substituted with other amino acids including proline.

As used herein, “biomolecule” refers to molecules having biological activity in a living body. In one embodiment of the present invention, the biomolecule may be selected from the group consisting of insulin, insulin analogue, glucagon, glucagon-like peptides, GLP-1 and glucagon dual agonist, GLP-1 and GIP dual agonist, GLP-1 and glucagon and GIP triple agonist, exendin-4, exendin-4 analogue, insulin secreting peptide and an analogue thereof, human growth hormone, growth hormone releasing hormone (GHRH), growth hormone releasing peptide, granulocyte colony stimulating factor (G-CSF), anti-obesity peptide, G-protein-coupled receptor, leptin, GIP (gastric inhibitory polypeptide), interleukins, interleukin receptors, interleukin binding proteins, interferons, interferon receptors, cytokine binding proteins, macrophage activator, macrophage peptide, B cell factor, Tcell factor, suppressive factor of allergy, cell necrosis glycoprotein, immunotoxin, lymphotoxin, tumor necrosis factor (TNF), tumor inhibitory factor, metastasis growth factor, alpha-1 antitrypsin, albumin, α-lactalbumin, apolipoprotein-E, erythropoietin (EPO), high glycosylated erythropoietin, angiopoietins, hemoglobin, thrombin, thrombin receptor activating peptide, thrombomodulin, blood factors VII, VIIa, VIII, IX, and XIII, plasminogen activator, fibrin-binding peptide, urokinase, streptokinase, hirudin, protein C, C-reactive protein, renin inhibitor, collagenase inhibitor, superoxide dismutase, platelet derived growth factor, epithelial growth factor, epidermal growth factor, angiostatin, angiotensin, bone morphogenetic growth factor, bone morphogenetic protein, calcitonin, atriopeptin, cartilage inducing factor, elcatonin, connective tissue activator, tissue factor pathway inhibitor, follicle stimulating hormone (FSH), luteinizing hormone (LH), luteinizing hormone releasing hormone (LHRH), nerve growth factors, parathyroid hormone (PTH), relaxin, secretin, somatomedin, adrenal cortical hormone, cholecystokinin, pancreatic polypeptide, gastrin releasing peptide, corticotropin releasing factor, thyroid stimulating hormone (TSH), autotaxin, lactoferrin, myostatin, receptor, receptor antagonist, fibroblast growth factor, adiponectin, interleukin receptor antagonist, cell surface antigen, virus derived vaccine antigen, monoclonal antibody, polyclonal antibody and antibody fragments.

As used herein, “binding site” refers to a site that is bound to another material or component, and “binding moiety” refers to a component including a portion capable of binding to another material or component. In addition, as used herein, “active site” refers to a site that induces activity by reacting with a ligand or receptor.

In addition, UCT (ubiquitin C-terminal tag) refers to a specific sequence of the C-terminal site of the ubiquitin, and UCT is conjugated with a specific lysine of another ubiquitin through covalent bond.

In addition, the present invention provides a method for preparing a multifunctional multispecific multimeric biomolecule polymer, in which a polyubiquitin scaffold, two or more biomolecules comprising binding moieties, each specific for different binding sites, and a carrier that prolongs the in vivo duration are directly bound to the N-terminus or the C-terminus of the ubiquitin or are bound by a linker, wherein the method comprises (i) recombinantly expressing a biomolecule to which a ubiquitin C-terminal tag is fused or bound by a linker from a host cell including a procaryotic cell or a eukaryotic cell, and (ii) adding E1, E2 and E3 enzymes, or E1 and E2 enzymes for ubiquitination to the cell lysates or purified products of the host cell and reacting them, wherein the polyubiquitin scaffold is formed by covalently bonding two or more ubiquitins, and the biomolecule is composed of 2 to 10 biomolecules, has active sites that specifically bind to other biomolecules, small molecule chemical compounds or nanoparticles, and is bound to the N-terminus, the C-terminus, or both the N-terminus and the C-terminus of the ubiquitin by a linker.

In one embodiment related thereto, the E2 enzyme may bind to the 6th, 11th, 27th, 29th, 33rd, 48th or 63rd lysine from the N-terminus of the ubiquitin, or may be an E2-25K ubiquitin conjugating enzyme or Ucb13-MMS2, a ubiquitin conjugating enzyme complex, but is not limited thereto.

In another embodiment related thereto, the ubiquitin C-terminal tag may be one in which two or more ubiquitins are repeatedly linked in a head-to-tail form or in a branched form (branched type or iso-peptide branch type form), wherein the ubiquitin linked in a head-to-tail form or in a branched form may be one in which the 75th and 76th glycines from the N-terminus are substituted with other amino acids including valine.

### Effects of the Invention

According to the present invention, since the linkage between biomolecule polymers or complexes is made by a polyubiquitin scaffold, the polyubiquitin may act as a rigid scaffold or linker that maintains the spacing and orientation between biomolecules bound to the polyubiquitin. Therefore, a multifunctional multispecific multimeric biomolecule polymer can be prepared without interference of the active site.

In addition, according to the present invention, the multifunctional multispecific multimeric biomolecule polymer is provided in a form that is bound to a molecule capable of increasing the in vivo duration, and thus may be used for producing drugs requiring the increased in vivo stability and duration of efficacy.

In the present invention, the biomolecule may be one or more selected from the group consisting of a protein, peptide, polypeptide, antibody, antibody fragment, DNA and RNA, and, for example, by using heterologous proteins, modularized functionality may be imparted to the linear multifunctional multimeric polymer. In addition, according to the present invention, the multifunctional multispecific multimeric biomolecule polymer is provided in a form that is bound to a molecule capable of increasing the in vivo duration, and thus may provide the increased stability and duration of efficacy in vivo for the biomolecule.

### BRIEF DESCRIPTION OF DRAWINGS

FIG. 1 shows the process of preparing the linear multifunctional multimeric fusion protein (UniStac) of the present invention.

FIGS. 2 and 3 show results of confirming the UCT fusion protein in a multimeric form formed by the UniStac reaction of the present invention.

FIG. 4 shows various application forms of the linear multifunctional multimeric fusion protein of the present invention.

FIG. 5 shows the results of UniStac preparation using only E1-E2.

FIG. 6 schematically shows the preparation of the linear multifunctional multimeric fusion protein of the present invention and the use thereof by immobilization.

FIG. 7 schematically shows the head-to-tail UCT and UniStac method.

FIG. 8 is a result of confirming by SDS-PAGE after purification of xylose reductase (XR) prepared according to the present invention by GPC.

FIG. 9 is a result of confirming by SDS-PAGE after purification of oxaloacetate decarboxylase (OAC) prepared according to the present invention by GPC.

FIG. 10 is a result of confirming by SDS-PAGE after purification of xylitol dehydrogenase (XDH) prepared according to the present invention by GPC.

FIG. 11 is a result of confirming by SDS-PAGE after purification of triose-phosphate isomerase (TIM) prepared according to the present invention by GPC.

FIG. 12 is a result of confirming by SDS-PAGE after purification of aldolase (ALD) prepared according to the present invention by GPC.

FIG. 13 is a result of confirming by SDS-PAGE after purification of fructose 1,6-bisphosphatase (FBP) prepared according to the present invention by GPC.

FIG. 14 is a result of confirming by SDS-PAGE after purification of pyruvate oxidase (POPG) prepared according to the present invention by GPC.

FIG. 15 is a result of analysis of the activity of xylose reductase.

FIG. 16 is a result of analysis of the stability of xylose reductase.

FIG. 17 is a result of analysis of the activity of oxaloacetate decarboxylase.

FIG. 18 is a result of analysis of the stability of oxaloacetate decarboxylase.

FIG. 19 is a result of analysis of the activity of xylitol dehydrogenase.

FIG. 20 is a result of analysis of the stability of xylitol dehydrogenase.

FIG. 21 is a result of analysis of the activity of pyruvate oxidase.

FIG. 22 shows the UniStac polymer of the structure to which three enzymes, TIM, ALD and FBP are bound.

FIG. 23 shows the synergistic effect by TIM, ALD and FBP enzymes.

FIG. 24 is a result of preparing and confirming Protein A and Protein G linear multifunctional multimer complexes.

FIG. 25 is a result of preparing and confirming hGH in which aspartate is extended at the C-terminal portion of the 76th glycine of the ubiquitin C-terminal tag.

FIG. 26 is a result of preparing and confirming the polymer originated from E3.

FIG. 27 is a result of preparing and confirming the polymer of hGH according to the presence or absence of DUB.

FIG. 28 shows that the binding activity of human derived IgG to the beads on which the Protein A monomer is immobilized and the beads on which the Protein A polymer is immobilized.

FIG. 29 shows the structure of a linear multifunctional multimeric biomolecule polymer bound to the N-terminus, the C-terminus, or both the N-terminus and the C-terminus of the ubiquitin, respectively, and a result of the preparation thereof.

FIG. 30 is a PK profile result showing that the half-life in blood is at the same level and the bioabsorption rate (AUC) is more excellent when comparing a biomolecule polymer in which carrier is bound to double ubiquitin and a human serum albumin.

FIG. 31 shows a pcDNA3.1 (+) vector to which a gene expressing an Fc based acceptor protein is linked.

FIG. 32 shows a result of confirming the expression of the Fc based acceptor protein.

FIG. 33 shows a result of confirming the expression of the Fc based acceptor protein that specifically binds to an IgG Fc antibody.

FIGS. 34 and 35 show results of purifying the Fc based acceptor protein.

FIG. 36 shows a result of confirming ubiquitin-IL-1RA through SDS-PAGE analysis method.

FIG. 37 shows the process of purifying ubiquitin-IL-IRA.

FIGS. 38 and 39 show results of purifying the His-SUMO tagged ubiquitin-IL-1RA protein.

FIG. 40 shows a result of confirming the His-SUMO tagged ubiquitin-IL-1RA through SDS-PAGE analysis method.

FIGS. 41 and 42 show results of confirming the His-SUMO detagged ubiquitin-IL-1RA.

FIGS. 43 and 44 show results of purifying the ubiquitin-IL-1RA protein.

FIG. 45 shows a result of confirming the degree of conjugation between the acceptor and the donor through SDS-PAGE analysis method.

FIG. 46 shows a result of analysis of the conjugation yield using μCE-SDS analysis method.

FIG. 47 shows the process of purifying the conjugation.

FIGS. 48 and 49 show results of confirming the conjugation purified with Ni-sepharose.

FIGS. 50 and 51 show results of confirming the purified conjugation.

FIGS. 52 and 53 are results of confirming the final UniStac polymer in SDS-PAGE (reducing and native conditions).

FIG. 54 shows a result of confirming the monomer using μCE-SDS analysis method.

FIG. 55 shows a result of confirming the monomer using SEC-HPLC.

FIG. 56 shows the structures of the donor protein (D-192), the UniStac protein (C-193), and the fusion protein (C-192; comparative group) using an acceptor protein using a human-serum albumin as a carrier.

FIG. 57 shows a graph of blood drug concentration over time after subcutaneous administration of the fusion proteins (C-192 and D-192).

FIG. 58 shows a graph of blood drug concentration over time after subcutaneous administration of the fusion proteins (C-193 and D-192).

### BEST MODE FOR CARRYING OUT THE INVENTION

In one embodiment, the present invention provides a method for preparing a multifunctional multispecific multimeric biomolecule polymer, in which a polyubiquitin scaffold, two or more biomolecules comprising binding moieties, each specific for different binding sites, and a carrier that prolongs the in vivo duration are directly bound to the N-terminus or the C-terminus of the ubiquitin or are bound by a linker, wherein the method comprises (i) recombinantly expressing a biomolecule to which a ubiquitin C-terminal tag is fused or bound by a linker from a host cell including a procaryotic cell or a eukaryotic cell, and (ii) adding E1, E2 and E3 enzymes, or E1 and E2 enzymes for ubiquitination to the cell lysates or purified products of the host cell and reacting them, wherein the polyubiquitin scaffold is formed by covalently bonding two or more ubiquitins, and the biomolecule has active sites that specifically bind to other biomolecules, small molecule chemical compounds or nanoparticles, and is bound to the N-terminus, the C-terminus, or both the N-terminus and the C-terminus of the ubiquitin by a linker.

In the present invention, an initiator that initiates the formation of a multifunctional multispecific multimeric biomolecule polymer or complex may be E3, E2, E1, a free ubiquitin, or a target substrate of E3. Here, the E2 enzyme may bind to the 48th or 63rd lysine of the ubiquitin, and the E2 enzyme may be an E2-25K ubiquitin conjugating enzyme, or a ubiquitin conjugating enzyme complex Ucb13-MMS2.

In the present invention, each of the biomolecules preferably binds to the N-terminus of the ubiquitin. In addition, the multimeric biomolecule polymer may be composed of 2 to 30 biomolecules.

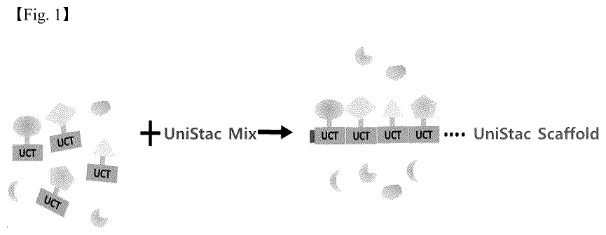

The UniStac reaction of the present invention is schematically shown in FIG. 1.

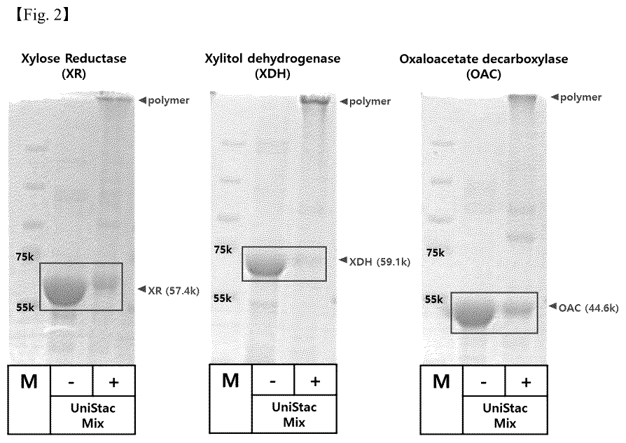

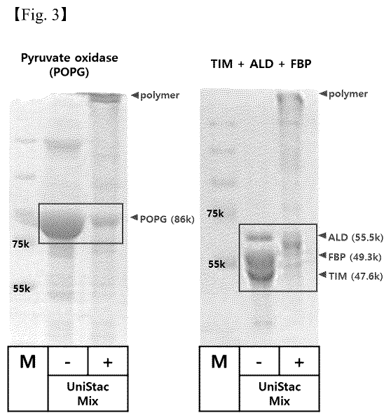

In addition, the results of confirming the UCT fusion protein in a multimeric form formed by the UniStac reaction are shown in FIGS. 2 and 3.

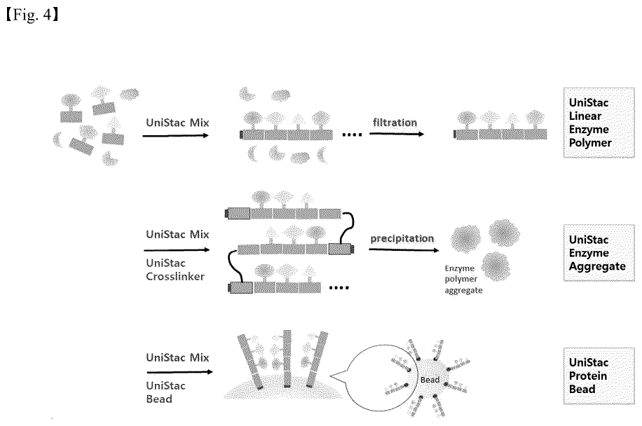

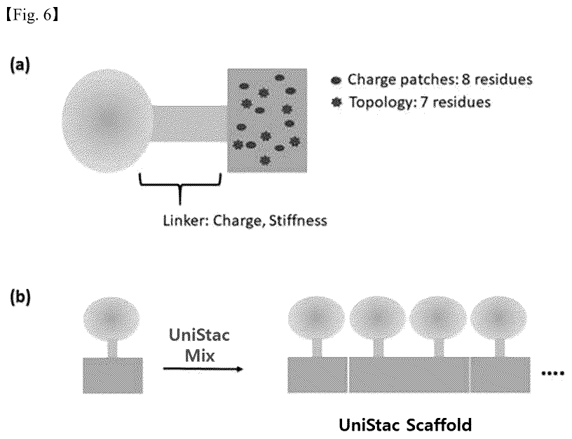

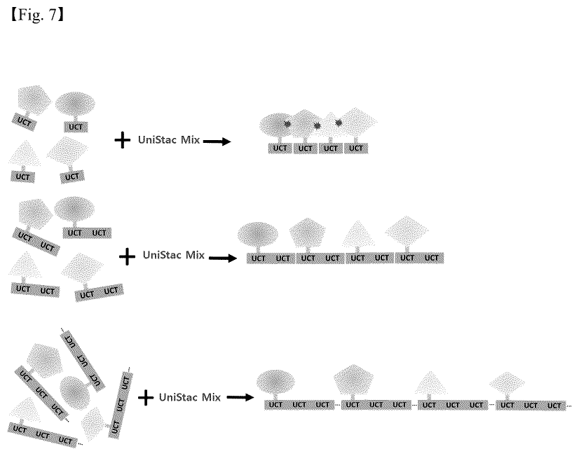

In addition, the multifunctional multispecific multimeric biomolecule polymer of the present invention may be fabricated in various forms. Specific examples are shown in FIGS. 4, 6 and 7. That is, the first drawing schematically shows a process of preparing a UniStac linear enzyme polymer by reacting a UniStac mixture with a ubiquitin C-terminal tagged enzyme as shown in FIG. 1 followed by filtration. The second drawing shows a process of preparing a UniStac enzyme aggregate by reacting the UniStac mixture with a ubiquitin C-terminal tagged enzyme, followed by precipitation with a cross linker. The third drawing schematically shows a process of immobilizing the ubiquitin C-terminal tagged protein onto a substrate or a bead.

Hereinafter, the present invention is to be described in more detail through the following examples. These examples are only for describing the present invention in more detail, and it will be apparent to those of ordinary skill in the art that the scope of the present invention is not limited by these examples according to the gist of the present invention.

### PREPARATION EXAMPLE

### Preparation Example 1: Cloning, Expression and Purification of C-Terminal Fusion Protein

The gene encoding the UCT (ubiquitin C-terminal tag) (SEQ ID NO: 1) protein fusion used in the examples of the present invention was produced on request by Genscript Inc.

In order to prepare a Ub out gene construct that does not comprise a ubiquitin tag at the C-terminus, fast cloning system (Li C, Wen A, Shen B, Lu J, Huang Y, Chang Y (2011). Fast cloning: a highly simplified, purification-free, sequence- and ligation-independent PCR cloning method. BMC Biotechnol 11, 92.) was used. This method is a technology capable of linking genes (insertion, removal or substitution) in which if the PCR product is directly treated with only Dpn1 in the absence of a restriction enzyme and ligase, Dpn1 plays a role of a restriction enzyme and ligase through a mechanism that has not yet been identified along with polymerase. In this method, using a primer designed to overlap both terminus with Phusion polymerase (Thermo Fisher Scientific), PCR (95° C. for 3 minutes, 95° C. for 15 seconds-55° C. for 1 minute-72° C. for 1 minute/kb 18 times repeated, 72° C. for 5 minutes, 12° C. for 20 minutes) was carried out on all vectors except for the region to be deleted. Next, the resulting PCR product was subjected to Dpn1 treatment for 1 hour at 37° C., and transformed into E. coli DH5a (Novagen), and then the plasmid of interest was obtained. All gene constructs were identified by commercial DNA sequencing.

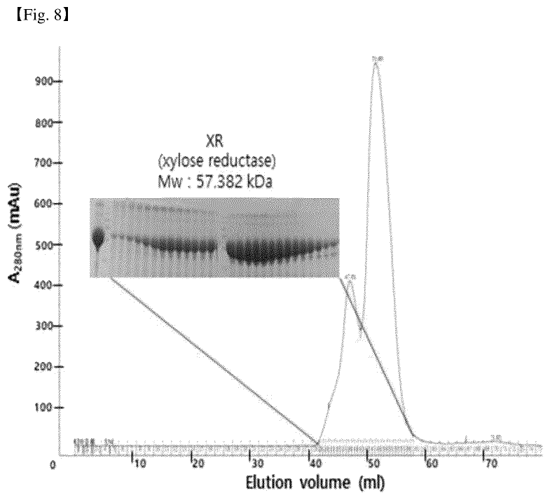

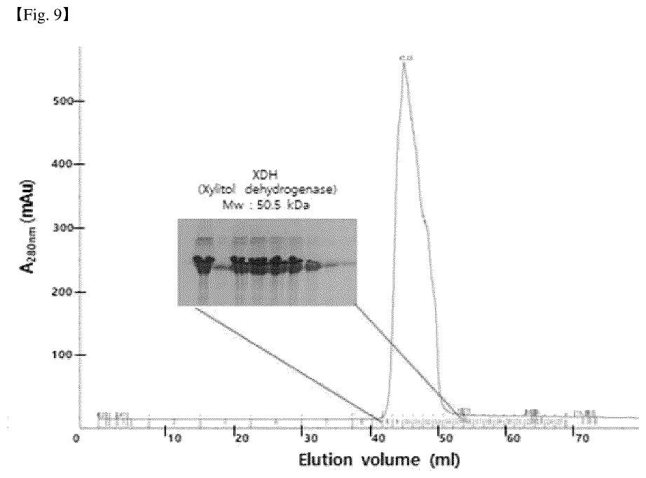

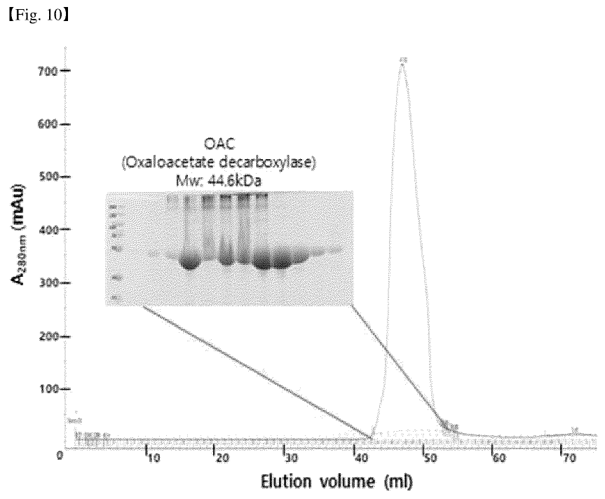

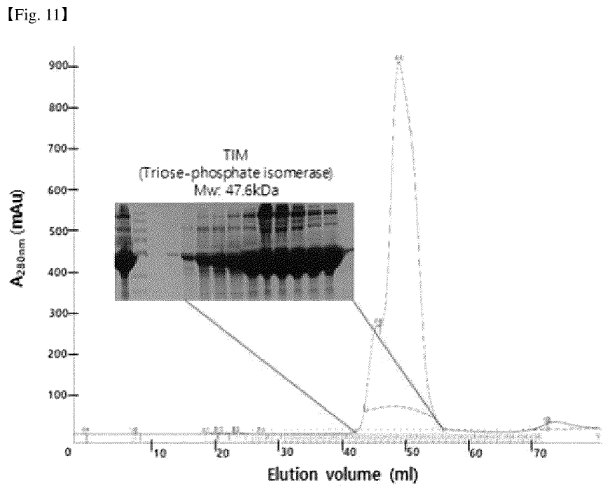

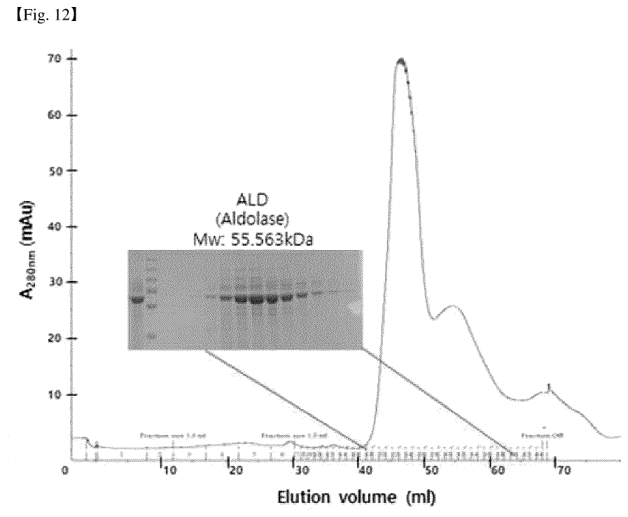

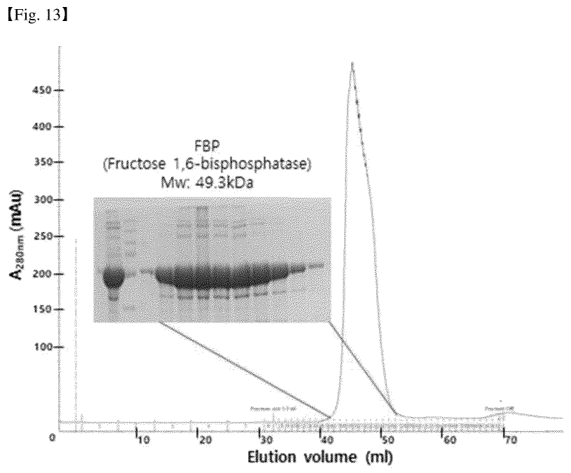

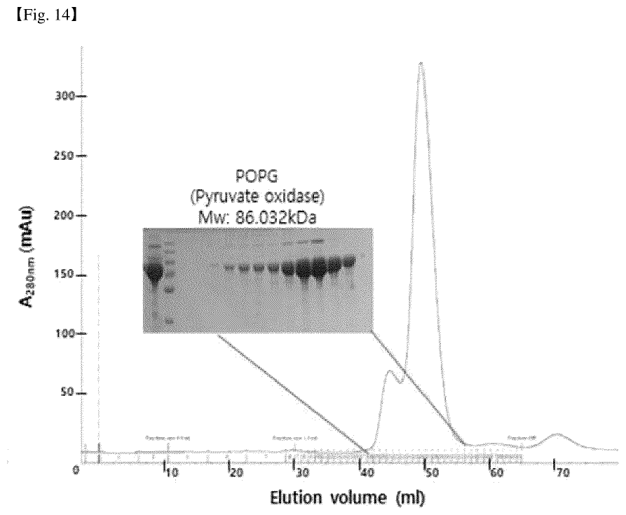

For overexpression of the UCT fusion protein, each gene construct was transformed into E. coli BL21 DE3 (Novagen) (XR, TIM, ALD), Rosetta pLysS DE3 (Novagen) (XDH, OAC, POPG), Origami2 DE3 (Novagen) (FBP) strains. Cells comprising the protein expression plasmid (pET21a, Genscript) were incubated in LB medium (Miller) at 37° C. When the OD₆₀₀ value reached about 0.6, the protein expression was induced with 250 μM of isopropyl β-D-1-thiogalactopyranoside (isopropyl-beta-D-thiogalactopyranoside) (IPTG) at 16° C. for 20 hours. Next, after centrifugation (at 3,500 rpm at 4° C. for 15 minutes), the cell pellet was resuspended in a lysis buffer (20 mM Tris-HCl pH 8.0, 500 mM NaCl₂, 20 mM imidazole) and lysed by sonication (50% amplitude, pluse on 3 seconds-off 5 seconds, final 15 minutes). Then, the lysate was further centrifuged at 14,000 rpm at 4° C. for 30 minutes. The water soluble fraction of the protein comprising the N-terminal His-tag was purified by gel filtration chromatography using Superdex 75 pg gel filtration column 16/600 (GE Healthcare) pre-equilibrated with nickel affinity and FPLC buffer (Ni-NTA Agarose, QIAGEN, 20 mM Tris-HCl pH 8.0, 150 mM NaCl₂). All UCT proteins were concentrated to 100 μM for analysis of the enzyme activity. All target proteins were evaluated by SDS-PAGE. FIGS. 8 to 14 show the results of confirming the target proteins. The UCT fusion proteins used in the present invention are shown in Table 1 below.

TABLE 1

| | Molecular weight | |
|---|---|---|
| UCT protein fusion | (kDa) | SEQ ID NO |
| Xylose reductase (XR) | 57.382 | SEQ ID NO: 2 |
| Xylitol dehydrogenase (XDH) | 59.1 | SEQ ID NO: 3 |
| Oxaloacetate decarboxylase (OAC) | 44.6 | SEQ ID NO: 4 |
| Triose-phosphate isomerase (TIM) | 47.6 | SEQ ID NO: 5 |
| Aldolase (ALD) | 55.563 | SEQ ID NO: 6 |
| Fructose 1,6-bisphosphatase (FBP) | 49.3 | SEQ ID NO: 7 |
| Pyruvate oxidase (POPG) | 86.032 | SEQ ID NO: 8 |

### Preparation Example 2: Preparation of UniStac Linear Construct

In the present invention, the reaction for preparing the fusion protein in a linear multifunctional multimeric form was designated as UniStac reaction. The UniStac reaction (a total volume of 50 μL) was carried out in the UniStac buffer (25 mM HEPES (Sigma-aldrich), pH 7.5, 50 mM NaCl, 4 mM MgCl₂), and the UniStac mixture for the UniStac reaction (0.5 μM E1, 5 μM E2, 1 μM E3, 4 mM ATP) was added to the UCT protein fusion of the present invention to initiate the reaction. The UniStac reaction was carried out by shaking at room temperature for 1 hour.

The ratio of proteins used in the reaction was at a concentration of 10 μM to 20 μM UCT protein fusion per 1 μM E3 enzyme (a ratio of 1:10 to 1:20), which was a condition set for the purpose so that at least 10 fusion monomers form a linear multifunctional multimer within 1 hour through the UniStac reaction. The E1, E2 and E3 used in the present invention are as follows, respectively:

TABLE 2

| Category | Name | SEQ ID NO |
|---|---|---|
| E1 | Yeast UBE1 | SEQ ID NO: 9 |
| E2 | Ubch5a [Homo sapiens] | SEQ ID NO: 10 |
| | (UniProtKB - P51668) | |
| | Ubch7 [Homo sapiens] | SEQ ID NO: 11 |
| | (UniProtKB - P68036) | |
| | E2-25K [Homo sapiens] | SEQ ID NO: 12 |
| | (UniProtKB - P61086) | |
| | Ubc13 [Saccharomyces cerevisiae] | SEQ ID NO: 13 |
| | (UniProtKB - P52490) | |
| | MMS2 (UEV—ubiquitin-conjugating | SEQ ID NO: 14 |
| | enzyme variant) | |
| E3 | RSP5 (UniProt ID. P39940) | SEQ ID NO: 15 |
| | DOA10 (UniProt ID. P40318) | SEQ ID NO: 16 |
| | MARCH5 (UniProt ID. Q9NX47) | SEQ ID NO: 17 |

### Preparation Example 3: Preparation of UniStac Using Only E1-E2 (E2 Platform)

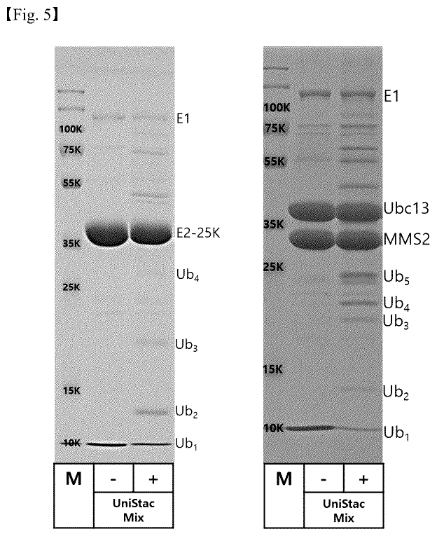

The E2-UniStac was prepared by using E2-25K (GenBank ID-U58522.1) (human E2), Ucb13 (yeast E2)-MMS2 (GenBank ID-U66724.1) (yeast ubiquitin-conjugating enzyme variant) (GenBank ID-U66724.1). The recombinant DNA plasmid synthesized by Genscript was used. The E2-UniStac reaction was carried out under a condition of the buffer (50 mM Tris pH 8.0, 5 mM MgCl₂), and the E2-UniStac mixture (1 μM E1, 10 μM E2, 4 mM ATP) was added to the free ubiquitin solution (20 μM) to initiate the reaction. The E2-UniStac reaction was carried out by shaking at room temperature for 1 hour. The results are shown in FIG. 5.

### EXAMPLE

### Example 1: Analysis of Activity and Stability of Xylose Reductase (XR)

### Analysis of Activity of Xylose Reductase

The UniStac reaction was carried out in the UniStac buffer (25 mM HEPES pH 7.5, 50 mM NaCl, 4 mM MgCl₂), and the UniStac mixture (0.5 μM E1, 5 μM E2, 1 μM E3, 4 mM ATP) was added to the XR protein solution to initiate the reaction. The UniStac reaction was carried out by shaking at room temperature for 1 hour, and then the catalytic activity was analyzed. The catalytic activity of XR was analyzed by measuring the change in absorbance at 340 nm induced by NADH oxidation.

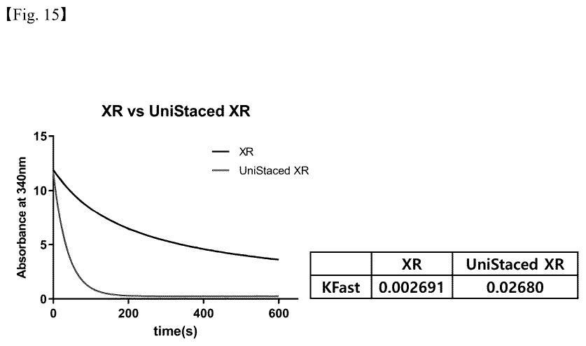

The reaction for analysis of the catalytic activity was initiated by adding NADH (2 mM) to a mixture of XR (10 μM) and xylose (200 mM) in a 100 mM NaCl buffer (pH 7.0) containing 1 mM MgCl₂ and 0.02% Tween-20. XR was a sample in the form of a monomer that did not comprise a ubiquitin tag at the C-terminus of the XR, and did not form a polymer under the same UniStac mixing condition. The statistical analysis was carried out using Prism 6 (GraphPad Software, Inc). The results are shown in FIG. 15.

As shown in FIG. 15, the XR according to the present invention promoted the reduction of D-xylose to xylitol by using NADH as a co-substrate. Absorbance represents the amount of NADH in solution. The UniStac polymer of the XR (lower curve) showed faster NADH consumption compared to the monomer form (upper curve). Both reactions contained an equal amount of the monomers. Therefore, the increased reaction rate is solely dependent on the covalent bonds between the monomers. In the end, it was confirmed that the activity of the XR UniStac polymer was increased by 10 times compared to the XR monomer without ubiquitin-tag

### Analysis of pH Stability of Xylose Reductase

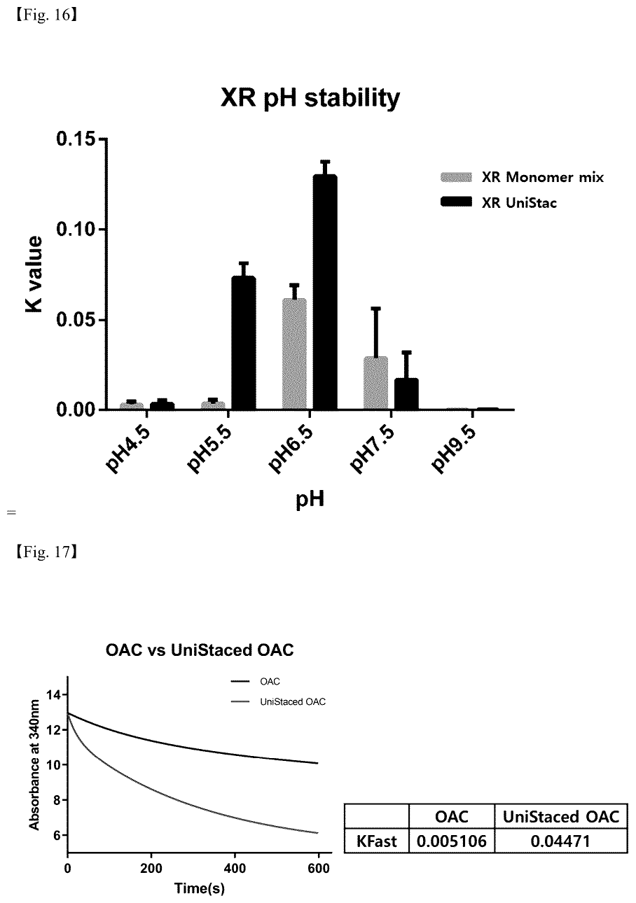

Both the XR monomer and the UniStac polymer were treated for 30 minutes at the indicated pH before initiating the reaction with the addition of NADH and xylose. As shown in FIG. 16, at pH 5.5 and 6.5, the XR UniStac polymer showed significantly enhanced stability compared to the XR monomer without ubiquitin-tag. The results represent the average value of the three experiments.

### Example 2: Analysis of Activity and Stability of Oxaloacetate Decarboxylase (OAC)

### Analysis of Activity of OAC

OAC involved in gluconeogenesis is used to investigate liver damage in conjunction with AST-ALT. The UniStac reaction was carried out in the UniStac buffer (25 mM HEPES pH 7.5, 50 mM NaCl, 4 mM MgCl₂), and the UniStac mixture (0.5 μM E1, 5 μM E2, 1 μM E3, 4 mM ATP) was added to the OAC protein solution to initiate the reaction. The UniStac reaction was carried out by shaking at room temperature for 1 hour, and then the catalytic activity was analyzed. The analysis of OAC activity was based on the decrease in absorbance (340 nm) as NADH consumption proceeded under the following conditions: 45 mM TEA buffer pH 8.0, 0.45 mM MnCl₂, 2 mM NADH, 11 U of LDH, 5 μM OAC, 2.5 mM.

The OAC was a sample in the form of a monomer that did not comprise a ubiquitin-tag at the C-terminus of the OAC, and did not form a polymer under the same UniStac mixing condition. The statistical analysis was carried out using Prism 6 (GraphPad Software, Inc). The results are shown in FIG. 17.

As shown in FIG. 17, as a result of comparing the activity of the monomer without Ub (OAC) at the C-terminus and the activity of the polymer (UniStaced OAC), the activity of the polymer was increased by 9 times. Absorbance represents the amount of NADH in solution. The UniStac polymer of the OAC (lower curve) showed faster NADH consumption compared to the monomer form (upper curve). Both reactions contained an equal amount of the monomers. Therefore, the increased reaction rate is solely dependent on the covalent bonds between the monomers. In the end, it was confirmed that the activity of the OAC UniStac polymer was increased by 9 times compared to the OAC monomer without ubiquitin-tag (OAC).

### Analysis of Stability of OAC

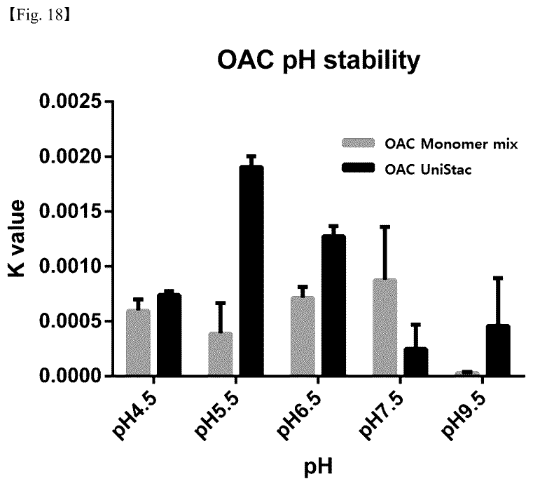

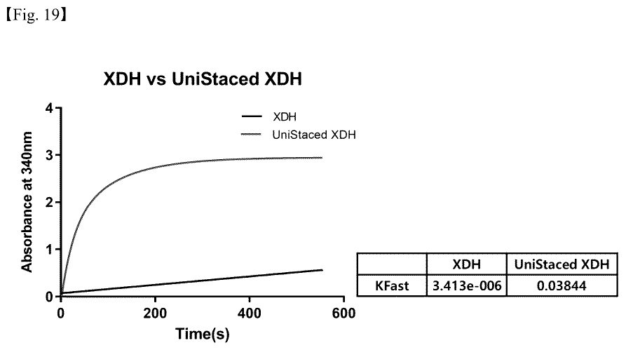

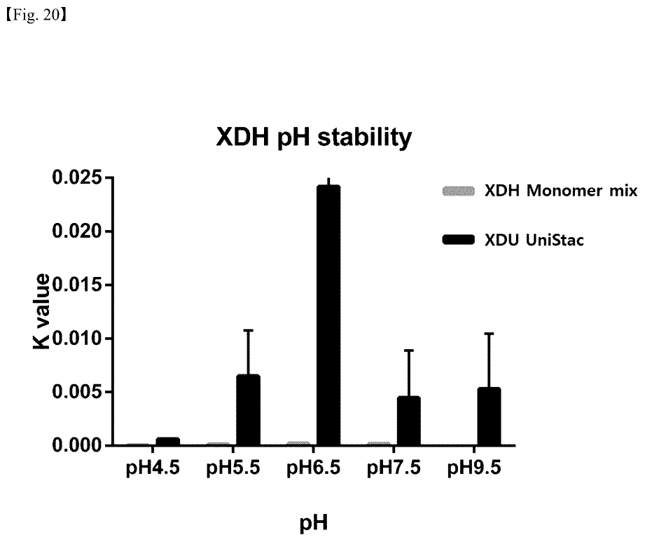

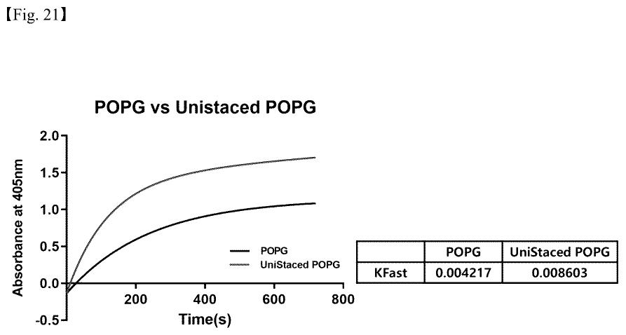

Both the OAC monomer and the UniStac polymer were treated for 30 minutes at the indicated pH before initiating the reaction with the addition of NADH and oxaloacetate. As shown in FIG. 18, at a low pH of pH 4.5 to 6.5, the OAC UniStac polymer showed significantly enhanced pH stability compared to the OAC monomer without ubiquitin-tag (OAC). The results represent the average value of the three experiments.

### Example 3: Analysis of Activity and Stability of Xylitol Dehydrogenase (XDH)

### Analysis of Activity of XDH

XDH is an enzyme belonging to the D-xylose catabolism pathway, and is known to convert xylitol, a product of XR, into xylulose using NAD⁺. For analysis of activity of XDH, the UniStac reaction was first carried out in the UniStac buffer (25 mM HEPES pH 7.5, 50 mM NaCl, 4 mM MgCl₂), and the UniStac mixture (0.5 μM E1, 5 μM E2, 1 μM E3, 4 mM ATP) was added to the XDH protein solution to initiate the reaction. The UniStac reaction was carried out by shaking at room temperature for 1 hour, and then the catalytic activity was analyzed. The activity of XDH was measured by monitoring NAD⁺ reduction at 340 nm. The reaction was initiated by adding NADH (2 mM) to a mixture of XDH (20 μM) and xylose (200 mM) in a 100 mM NaCl buffer (pH 7.0) containing 1 mM MgCl₂ and 0.02% Tween-20. The XDH was a sample in the form of a monomer that did not comprise a ubiquitin-tag at the C-terminus of the XDH, and did not form a polymer under the same UniStac mixing condition. The statistical analysis was carried out using Prism 6 (GraphPad Software, Inc). The results are shown in FIG. 19.

As shown in FIG. 19, at pH 5.5, the UniStac polymer of the XDH (upper curve) showed higher NADH+ consumption rate compared to its monomer form (lower curve). Both reactions contained an equal amount of the monomers. Therefore, the difference in activity is solely dependent on the covalent bonds between the monomers. In the end, it was confirmed that the activity of the XDH UniStac polymer was increased by 10 times compared to the XDH monomer without ubiquitin-tag (XDH).

### Analysis of Stability of XDH

Both the XDH monomer and the UniStac polymer were treated for 30 minutes at the indicated pH before initiating the reaction with the addition of NAD⁺ and xylitol. As shown in FIG. 20, at all measured pHs, the XR UniStac polymer showed significantly increased pH stability compared to the XDH. The results represent the average value of the three experiments.

### Example 4: Analysis of Activity of Pyruvate Oxidase (POPG)

POPG is known to be used to investigate liver damage by detecting enzymes such as AST-ALT, an enzyme involved in the gluconeogenesis process. For analysis of activity of POPG, the UniStac reaction was first carried out in the UniStac buffer (25 mM HEPES pH 7.5, 50 mM NaCl, 4 mM MgCl₂), and the UniStac mixture (0.5 μM E1, 5 μM E2, 1 μM E3, 4 mM ATP) was added to the POPG protein solution to initiate the reaction. The UniStac reaction was carried out by shaking at room temperature for 1 hour, and then the catalytic activity was analyzed. In order to analyze the catalytic activity, the amount of H₂O₂ produced by the POPG oxidation process of pyruvate by ABTS was measured. The reaction was initiated by adding POPG (5 μM) to a mixture of pyruvate (100 mM), pyrophosphate (6 mM), ABTS (2,2′-azino-bis(3-ethylbenzothiazoline-6-sulfonic acid) (10 mM) and HRP (horseradish peroxidase) (0.2 U/mL) in a sodium phosphate buffer.

The POPG monomer (POPG) was a sample in the form of a monomer that did not comprise a ubiquitin tag at the C-terminus of the POPG, and did not form a polymer under the same UniStac mixing condition. The statistical analysis was carried out using Prism 6 (GraphPad Software, Inc). As shown in FIG. 21, at pH 5.5, the POPG (upper curve) showed higher activity compared to its monomer form (lower curve). Both reactions contained an equal amount of the monomers. Therefore, the difference in activity is solely dependent on the covalent bonds between the monomers. In the end, it was confirmed that the activity of the POPG UniStac polymer was increased by 2 times compared to the POPG monomer without ubiquitin-tag (POPG).

### Example 5: Analysis of Synergistic Effect of Ubiquitin Enzyme

Triosephosphate isomerase (TIM), fructose bisphosphate aldolase (ALD) and fructose bisphosphatase (FBP) are known to form a cascade reaction for producing F6P as a final product from DHAP (dihydroxyacetone phosphate). The analysis of synergistic effect of the UniStac enzyme was carried out by measuring fructose-6-Phosphate (F6P), a TIM product, ALD and FBP enzyme complex. F6P is isomerized to glucose-6-phosphate (G6P) by phosphoglucose isomerase (PGI), and an equal amount of NAD⁺ as a substrate is modified by glucose-6-phosphate dehydrogenase (G6PDH). The present inventors determined the enzyme activity by measuring the amount of newly generated NADH at 340 nm by adding 2.5 mM enzyme complex (dihydroxyacetone phosphate, DHAP), 20 U/mL analysis enzyme (PGI and G6PDH) and 2.5 mM NAD⁺ enzyme complex to a mixture of 4 μM TIM, ALD and FBP enzyme complex in a HEPES buffer condition (200 mM HEPES pH 7.5, 10 mM MgCl₂, 0.5 mM MnCl₂, 1 mM CaCl₂)).

The enzyme complex mixture was a sample in the form of a monomer that did not comprise a ubiquitin tag at the C-terminus of the enzyme, and did not form a polymer under the same UniStac mixture condition. The statistical analysis was carried out using Prism 6 (GraphPad Software, Inc). At the indicated time point, the reaction was terminated, and the amount of F6P was measured using a phosphoglucose isomerase (PGI) that uses NAD⁺ to convert F6P into glucose-6-phosphate (G6P). Absorbance represents the amount of F6P.

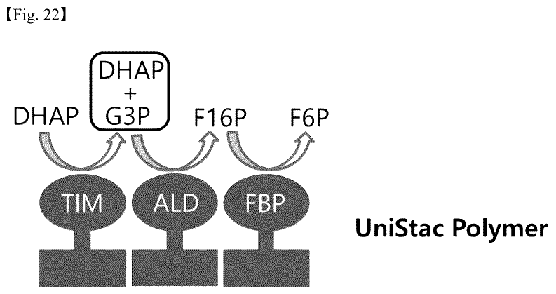

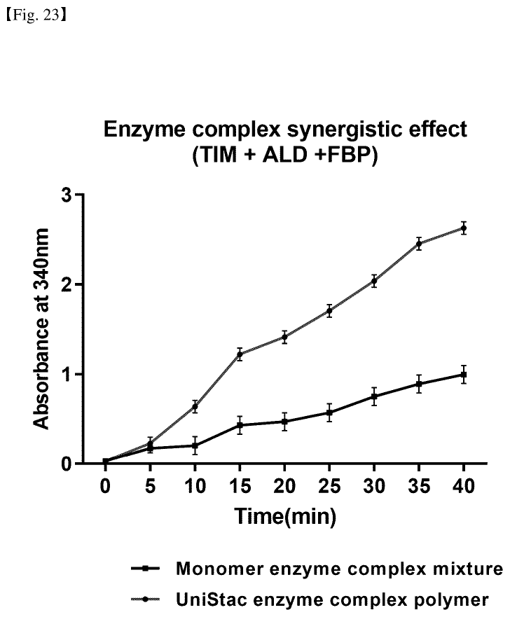

The results of the experiment are shown in FIG. 23. As shown in FIG. 23, the UniStac polymer of three different enzymes (upper curve) showed higher activity by five times than the monomeric enzyme mixture (lower curve), confirming the synergistic effect by the UniStac enzymes. FIG. 22 shows the resulting product of the structure (UniStac Polymer) in which three enzymes, TIM, ALD and FBP are bound.

### Example 6: Ubiquitin Multistage Labeling (Prosthetics) Method

A ubiquitin C-terminal tagged biomolecule was synthesized according to the preparation examples of the present invention. Next, a polymer (polyethylene glycol) comprising hydroxylamine was reacted with the above biomolecule. As a result, it was confirmed that the polymer was labeled with ubiquitin by an oxime linkage. The oxime linkage can be used as a tool capable of allowing a polymeric drug delivery system.

### Example 7: Preparation of Protein A and Protein G Linear Multimeric Polymer

The UniStac reaction (a total volume of 50 μL) was carried out in the UniStac buffer (25 mM HEPES pH 7.5, 50 mM NaCl, 4 mM MgCl₂), and the UniStac mixture (0.5 μM E1, 5 μM E2, 1 μM E3, 4 mM ATP) was added to the Protein A or Protein G solution to initiate the reaction. Recombinant DNA plasmids comprising sequences corresponding to Protein A (GenBank ID-AAB05743.1) and Protein G (CAA27638.1) that were synthesized by Genscript were used. The UniStac reaction was carried out by shaking at room temperature for 1 hour, and then SDS-PAGE was carried out.

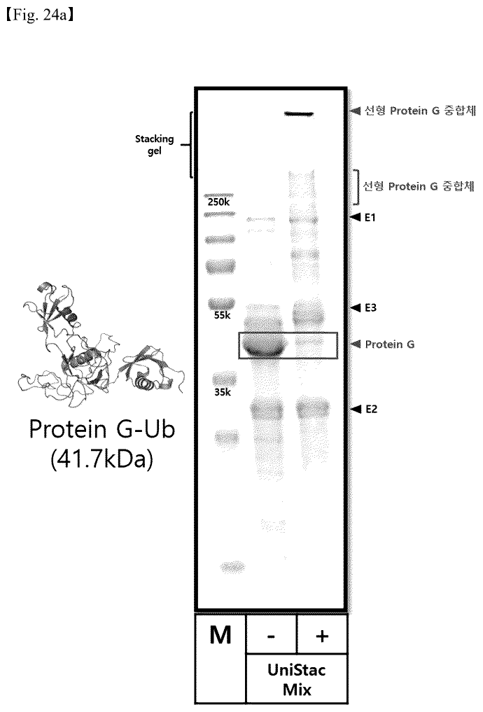

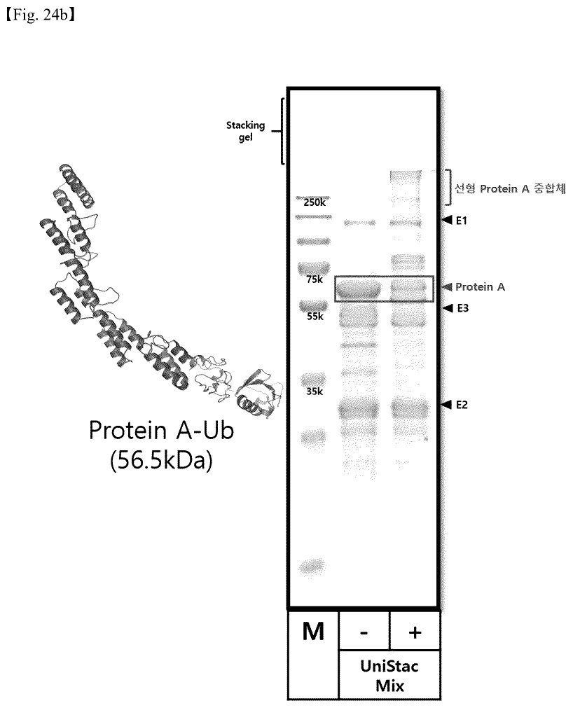

As shown in FIG. 24, compared to the sample without the UniStac mixture, it was confirmed that a monomer band of Protein A or Protein G was reduced in the sample to which the UniStac mixture was added, and a band of high molecular weight (linear multimeric polymer) newly appeared. In addition, it was confirmed that some linear multimeric polymers did not pass through a stacking gel due to an increase in molecular weight of up to several hundreds kDa.

### Example 8: hGH in which the C-Terminus of the 76th Glycine of the Ubiquitin C-Terminal Tag is Extended by Aspartate

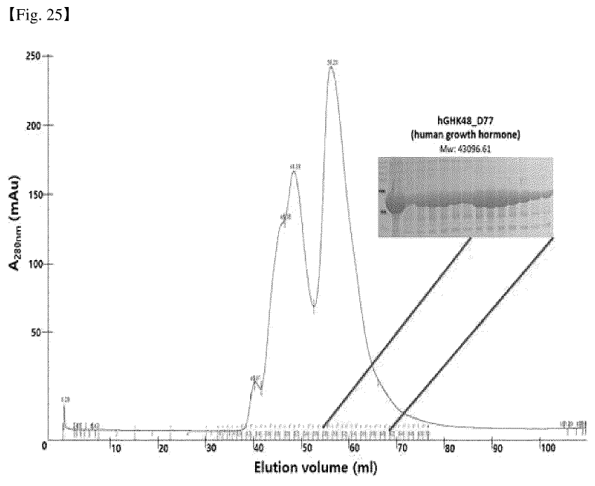

For overexpression of the UCT fusion protein, each gene construct was transformed into E. coli BL21 DE3 (Novagen) strain. In this example, hGH (SEQ ID NO: 18) was used as a protein. Cells comprising the protein expression plasmid (pET21a, Genscript) were incubated in LB medium (Miller) at 37° C. When the OD₆₀₀ value reached about 0.6, the protein expression was induced with 250 μM isopropyl β-D-1-thiogalactopyranoside (isopropyl-beta-D-thiogalactopyranoside) (IPTG) at 16° C. for 20 hours. Next, after centrifugation (at 3,500 rpm at 4° C. for 15 minutes), the cell pellet was resuspended in a lysis buffer (20 mM Tris-HCl pH 8.0, 500 mM NaCl₂, 20 mM imidazole) and lysed by sonication (50% amplitude, pluse on 3 seconds-off 5 seconds, final 15 minutes). Then, the lysate was further centrifuged at 14,000 rpm at 4° C. for 30 minutes. The water soluble fraction of the protein comprising the N-terminal His-tag was purified by gel filtration chromatography using Superdex 75 pg gel filtration column 16/600 (GE Healthcare) pre-equilibrated with nickel affinity and FPLC buffer (Ni-NTA Agarose-QIAGEN, 20 mM Tris-HCl pH 8.0, 150 mM NaCl₂). The purified hGH was concentrated to 100 μM and evaluated by SDS-PAGE. The results are shown in FIG. 25.

### Example 9: Preparation of Polyubiquitin Scaffold Originated from E3 (Rsp5)

The UniStac reaction (a total volume of 50 μL) was carried out in the UniStac buffer (25 mM HEPES pH 7.5, 50 mM NaCl, 4 mM MgCl₂), and the UniStac mixture (0.5 μM E1, 5 μM E2 (Ubch5a or Ubch7), 1 μM E3, 4 mM ATP) was added to the protein solution to initiate the reaction. The UniStac reaction was carried out by shaking at room temperature for 1 hour, and then SDS-PAGE was carried out.

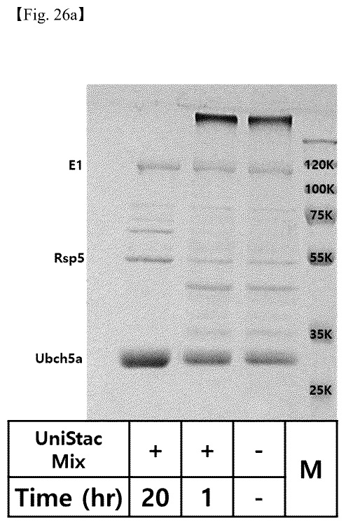

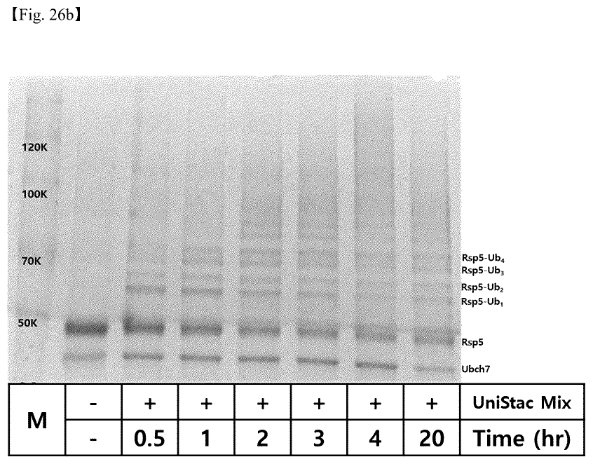

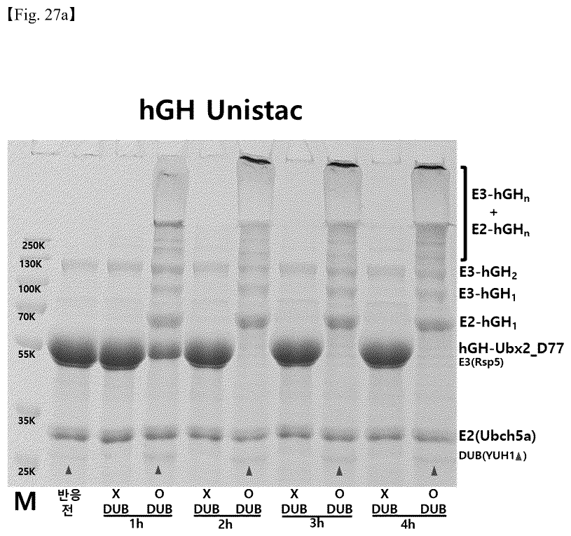

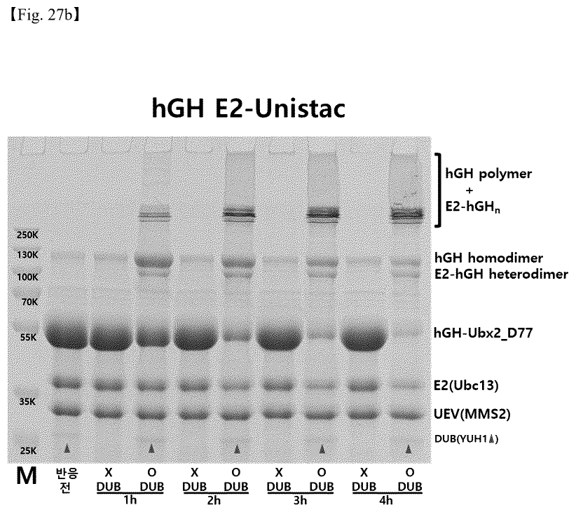

As shown in FIG. 26, it was confirmed that the amount of E3 was reduced in the sample to which the UniStac mixture was added compared to the sample without the UniStac mixture. This is because the band of E3 whose molecular mass was increased due to the formation of a polyubiquitin scaffold originated from E3 was shifted upwards. In addition, compared to the UniStac mixture comprising Ubch5a E2 (FIG. 26a), in the results of adding the UniStac mixture comprising Ubch7 E2 that has a weak reactivity (FIG. 26b), it was confirmed that the molecular weight was gradually increased by linking ubiquitin one by one to E3 (Rsp5) over time.

### Example 10: Preparation of Polymer of hGH According to the Presence or Absence of DUB

The UniStac reaction of hGH (SEQ ID NO: 18) was compared under the condition where DUB was present together and the condition where DUB was excluded. The hGH used at this time is one in which two ubiquitin tags are repeatedly connected at the C-terminus in a head-to-tail form, and the C-terminus of the ubiquitin tag is extended with aspartate. Therefore, if the aspartate at the C-terminus of the ubiquitin tag is not cleaved using DUB, the UniStac reaction does not occur.

The UniStac reaction to confirm the polymer formation of hGH, a biomolecule was carried out in the UniStac buffer (25 mM HEPES pH 7.5, 50 mM NaCl, 4 mM MgCl₂), and the UniStac mixture (1 μM E1, 5 μM E2 (ubch5a), 1 μM E3, 4 mM ATP) was added to 20 μM hGH protein solution to initiate the reaction.

In addition, the E2-UniStac reaction where E3 was excluded was carried out in the E2-UniStac buffer (50 mM Tris pH 8.0, 5 mM MgCl₂), and the E2-UniStac mixture (1 μM E1, 10 μM E2 (Ucb13-MMS2 complex), 4 mM ATP) was added to 20 μM hGH protein solution to initiate the reaction.

In order to confirm the activity of DUB together, the reaction was carried out simultaneously under the condition where DUB (YUH1) was absent and the condition where DUB (YUH1) was present at a concentration of 2 μM, respectively. All reactions were carried out by shaking at room temperature for 1 to 4 hours and then confirmed by SDS-PAGE.

As shown in FIG. 27, it was confirmed that the polymer of hGH was formed only under the condition where DUB was present, and it was confirmed that the polymer was not formed because the aspartate at the C-terminus of the hGH UCT was not cleaved under the condition where DUB was absent.

### Example 11: Binding Activity of Protein A Polymer Immobilized on Bead

The UniStac reaction to prepare the Protein A polymer was carried out in the UniStac buffer (25 mM HEPES pH 7.5, 50 mM NaCl, 4 mM MgCl₂). The UniStac mixture (0.5 μM E1, 5 μM E2 (Ubch5a or Ubch7), 1 μM E3, 4 mM ATP) was added to the Protein A protein solution to initiate the reaction. The UniStac reaction was carried out by shaking at room temperature for 1 hour, and then mixing in a 1:1 ratio together with latex beads at 50% concentration, and then shaking at ambient temperature for 4 hours, and immobilizing the Protein A polymer on the beads. After the immobilization reaction, in order to remove the unimmobilized protein, washing was carried out three times with the PBS buffer (10 mM Na₂HPO₄ PH 7.4, 1.8 mM KH₂PO₄, 137 mM NaCl, 2.7 mM KCl). After washing, the immunoglobulin G (IgG) obtained from human serum was added to the beads at a concentration of 2 mg/mL to analyze the binding activity of the Protein A polymer immobilized on the beads. The binding reaction was carried out by shaking at ambient temperature for 1 hour, and then washing three times with the PBS buffer in the same manner as in the above washing method, and then confirmed by SDS-PAGE.

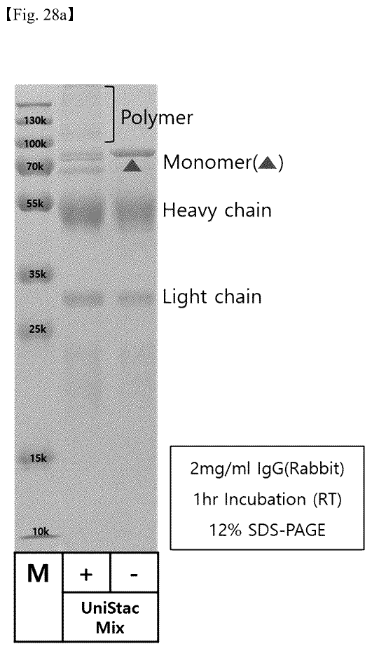

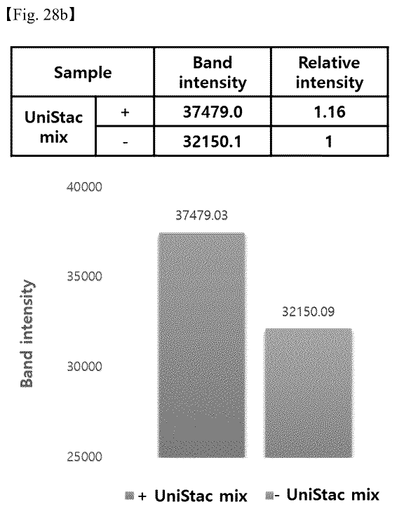

As shown in FIG. 28, it was confirmed that the binding activity of human derived IgG to the beads on which the Protein A polymer was immobilized was increased by 15% or more compared to the beads on which the Protein A monomer was immobilized in the same manner except that the UniStac mixture was not added.

### Example 12: Preparation of Linear Multivalent Biomolecule Polymer Bound to N-Terminus, C-Terminus, or Both N-Terminus and C-Terminus of Ubiquitin, Respectively

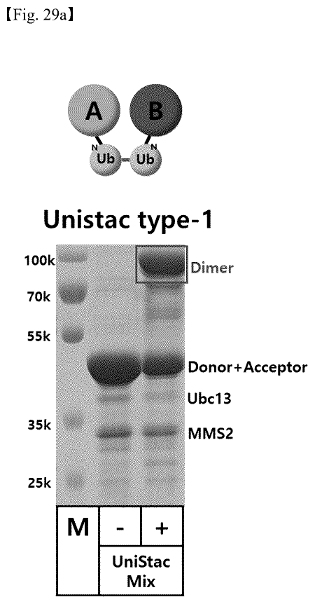

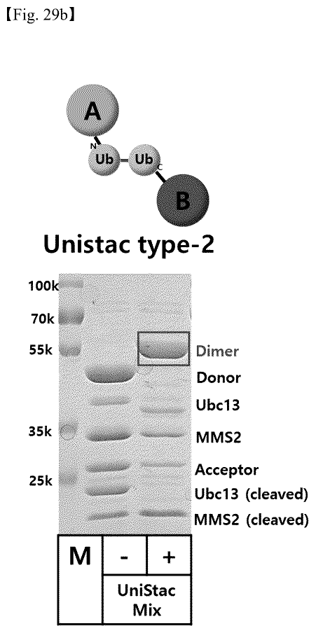

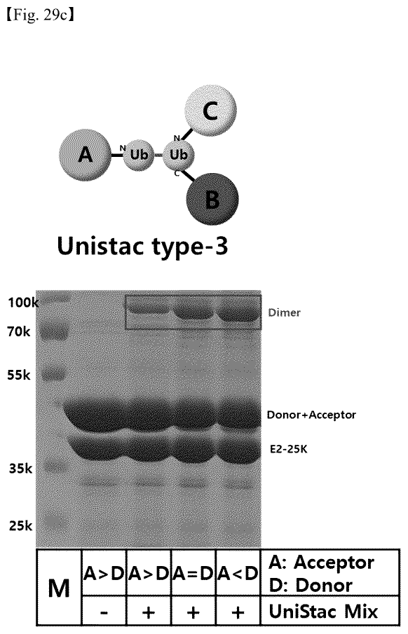

The formation of the UniStac dimer was confirmed by preparing the dimer of the donor ubiquitin in which hGH was bound to the N-terminus (SEQ ID NO: 18) and the acceptor ubiquitin in which hGH was bound to the N-terminus (SEQ ID NO: 19) (FIG. 29 (a)); the dimer of the donor ubiquitin in which hGH was bound to the N-terminus (SEQ ID NO: 18) and the acceptor ubiquitin in which hGH was bound to the C-terminus (SEQ ID NO: 20) (FIG. 29 (b)); and the dimer of the donor ubiquitin in which hGH was bound to the N-terminus (SEQ ID NO: 18) and the acceptor ubiquitin in which SUMO and hGH were bound to the N-terminus and the C-terminus, respectively (SEQ ID NO: 21) (FIG. 29 (c)), respectively.

The acceptor ubiquitin is a form in which the 73rd leucine is substituted with proline, a form in which other lysines except for the 48th lysine (FIG. 29 (c)) or the 63rd lysine (FIG. 29 (a) and (b)) of the acceptor ubiquitin are substituted with arginine, and a form in which the C-terminus is extended by aspartate or biomolecule (hGH).

The UniStac reaction (FIG. 29 (a) and (b)) was carried out in the UniStac buffer (25 mM HEPES pH 7.5, 50 mM NaCl, 4 mM MgCl₂), and the UniStac mixture (1 μM E1, 5 μM E2 (Ubc13-MMS2 complex), 4 mM ATP) was added to a solution (a total ubiquitin concentration of 20 μM) in which 10 μM acceptor ubiquitin protein and donor ubiquitin protein were mixed to initiate the reaction.

In addition, the UniStac reaction (FIG. 29 (c)) was initiated by replacing E2 with E2-25K other than Ubc13-MMS2 complex, and the acceptor ubiquitin with a protein having only the 48th Lys other than the 63rd Lys under the same conditions as the above reaction. The UniStac reaction was carried out by shaking at 27° C. for 4 hours and then confirmed by SDS-PAGE. In the UniStac reaction (FIG. 29 (b)), the acceptor ubiquitin in the Ub-hGH form in which His-sumo was cleaved using the SENP1 enzyme from a protein in the His-sumo-Ub-hGH form was used, and it was confirmed that at this time, the remaining SENP1 was included in the UniStac reaction, and thus the donor hGH, Ubc13 and His-sumo of MMS2 were also cleaved together, and the band of dimer and E2 (Ubc13, MMS2) after reaction was shifted.

As shown in FIG. 29, it was confirmed that the UniStac dimer was formed in all forms in which the biomolecule was bound to the N-terminus, the C-terminus, or both the N-terminus and the C-terminus, respectively. SEQ ID NOs of proteins and the like used in this example are as follows: the donor ubiquitin in which hGH was bound to the N-terminus (SEQ ID NO: 18); the acceptor ubiquitin in which hGH was bound to the N-terminus (SEQ ID NO: 19); the acceptor ubiquitin in which hGH was bound to the C-terminus (SEQ ID NO: 20); the acceptor ubiquitin in which SUMO and hGH were bound to the N-terminus and the C-terminus, respectively (SEQ ID NO: 21).

### Example 13: Confirmation of Pharmacokinetics

Diubiquitin-albumin (OGB1) and albumin (OGB3) were subcutaneously administered to 9-week-old male Spraque-Dawley rats once, and then blood was collected for each time period, and the drug concentration in the serum was analyzed. Diubiquitin-albumin was administered at 0.833 mg/kg, and albumin was administered at 1 mg/kg, and each group was composed of 12 male rats. Blood collection for analysis of blood drug concentration was carried out from 3 rats per group before administration (blank) and 0.5, 1, 2, 4, 6, 8, 12, 24, 36, 48 and 72 hours after administration (a total of 12 time points).

The control group was composed of 5 male rats, and blood collection was carried out 1 and 24 hours after administration. The serum was separated from the collected blood and was stored in a cryogenic frozen state at −70±10° C. Analysis was carried out by measuring drug concentrations from the blood samples collected for each time period by the human serum albumin ELISA kit. Serum for dilution was prepared by mixing a buffer for dilution (1×PBS, 1% BSA) and a rat blank serum in a 1:1 ratio and used for the ELISA analysis. Diubiquitin-albumin was diluted in the serum for dilution from 800 ng/mL to 15.625 ng/mL and dispensed into each well. Each sample was diluted using a rat blank serum and a buffer for dilution to obtain a serum for dilution in a final ratio of 1:1 and dispensed into each well. An antibody mixture solution was prepared by diluting the Capture and Detector antibodies of the human serum albumin kit in the antibody dilution CP solution. 100 μL of the antibody mixture solution was dispensed into each well and incubated at 400 rpm at room temperature for 1 hour. When the incubation was completed, 300 μL of the washing solution was dispensed into each well, and the process of shaking was repeated three times. 100 μL of the TMB substrate solution was dispensed into each well and incubated at 400 rpm at room temperature for 10 minutes, and 100 μL of the stop solution was dispensed into each well and put into the instrument, and absorbance (OD450) was measured. Albumin was diluted from 100 ng/ml to 2.5 ng/ml and dispensed into each well, and the rest of the sample dilution and experimental procedure were performed in the same manner to measure absorbance. A calibration curve was calculated with 4 parameters based on the absorbance values measured for each concentration, and the drug concentration in the serum was finally calculated based on the absorbance values measured from the sample compared to the calibration curve. Pharmacokinetic parameters were calculated using Phoenix WinNonlin (Ver. 8.1, Pharsight-A Certara company, U.S.A.) for the results of measuring the concentration of the test substance in serum to evaluate the pharmacokinetics.

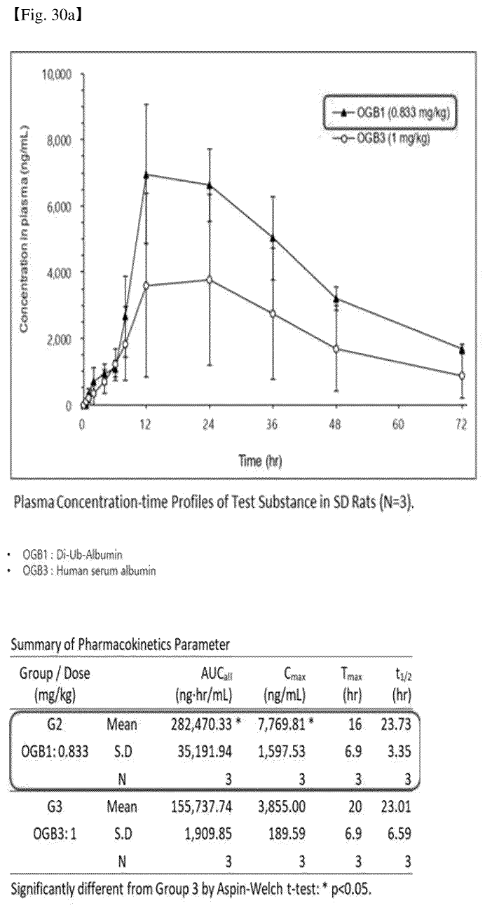

As shown in FIG. 30, it was confirmed that diubiquitin-albumin (OGB1) exhibited a higher plasma concentration. In addition, it was confirmed that AUC was increased by 1.8 times and Cmax was increased by more than 2 times in the group administered with diubiquitin-albumin (OGB1) compared to the group administered with albumin (OGB3). Therefore, it can be seen that the diubiquitin-albumin polymer of the present invention has a more excellent pharmacokinetic effect at a lower concentration.

In the end, the biomolecule polymer of the present invention is provided in a form that is bound to a molecule capable of increasing the in vivo duration, and thus may be used for producing a pharmaceutical composition requiring the increased in vivo duration of efficacy.

### Example 14: Preparation of Recombinant Expression Plasmid DNA Capable of Expressing Fc Based Acceptor Protein (Acceptor Protein)

A fusion protein in which a carrier was directly bound to the C-terminus of the acceptor ubiquitin was prepared by the following method. An antibody fragment (IgG Fc domain) was used as a carrier protein of the fusion protein, and the fusion protein is referred to as an “Fc based acceptor protein.”

A signal peptide that causes a protein to be secreted out of cells to express solubility in cells, an acceptor ubiquitin, a hinge and a Fc gene were designed to be inserted into the pcDNA3.1 (+) vector. The pcDNA3.1 (+) vector is an expression vector for animal cells having a CMV promoter, an ampicillin resistance gene, and the like.

FIG. 31 shows the pcDNA3.1 (+) vector to which a gene expressing an Fc based acceptor protein is linked. The nucleotide sequence and amino acid sequence expressing the Fc based acceptor protein are shown in Tables 3 and 4 below.

TABLE 3
Nucleotide sequence of IgGκ (SP)-Fc based acceptor protein

| | |
|---|---|
| Signal peptide (IgGκ) | ATGGAAACTG ATACTCTGCT GCTGTGGGTG CTGCTGCTGT |
| (SEQ ID NO: 22) | GGGTGCCCGG CTCAACTGGT |
| Ub (A) acceptor | ATGCAGATCT TCGTGAGGAC CCTGACAGAT |
| ubiquitin | CGGACCATCA CACTGGAGGT GGAGCCAAGC |
| (SEQ ID NO: 23) | GACACCATCG AGAACGTGAG GGCCAGAATC |
| | CAGGACCGGG AGGGCATCCC CCCTGATCAG |
| | CAGAGACTGA TCTTCGCTGG CCGCCAGCTG |
| | GAGGACGGAA GGACCCTGAG CGATTACAAT |
| | ATCCAGAAAG AGTCTACACT GCACCTGGTG |
| | CTGAGACCGC GCGTCGTGGA T |
| Hinge (IgG1) (SEQ | GAGCCAAAAT CTTGTGACAA AACTCATACA TGTCCC |
| ID NO: 24) | |
| Fc (IgG1) (SEQ ID | CCATGTCCCG CACCTGAACT GCTGGGCGGA |
| NO: 25) | CCTAGCGTGT TTCTGTTCCC ACCTAAGCCA AAGGACACCC |
| | TGATGATCTC CAGGACCCCC GAGGTGACAT |
| | GCGTGGTGGT GGACGTGAGC CACGAGGACC |
| | CCGAGGTGAA GTTCAACTGG TACGTGGATG |
| | GCGTGGAGGT GCATAATGCC AAGACAAAGC |
| | CAAGGGAGGA GCAGTACAAC AGCACCTATC |
| | GGGTGGTGTC TGTGCTGACA GTGCTGCACC |
| | AGGACTGGCT GAACGGCAAG GAGTATAAGT |
| | GCAAGGTGTC TAATAAGGCC CTGCCCGCTC |
| | CTATCGAGAA GACCATCTCC AAGGCCAAGG |
| | GCCAGCCAAG AGAGCCCCAG GTGTACACAC |
| | TGCCCCCTAG CCGCGACGAG CTGACCAAGA |
| | ACCAGGTGTC TCTGACATGT CTGGTGAAGG GCTTCTATCC |
| | TTCTGATATC GCTGTGGAGT GGGAGTCCAA |
| | TGGCCAGCCA GAGAACAATT ACAAGACCAC |
| | ACCACCCGTG CTGGACTCTG ATGGCTCCTT CTTTCTGTAT |
| | TCCAAGCTGA CCGTGGATAA GAGCAGATGG |
| | CAGCAGGGCA ACGTGTTCTC CTGTAGCGTG |
| | ATGCATGAAG CACTCATATAA TCACTATACC CAGAAGTCAC |
| | TGTCACTGAG TCCCGGTAAA |

TABLE 4
Amino acid sequence of IgGκ (SP)-Fc based acceptor protein

| | |
|---|---|
| Signal peptide (IgGκ) | Met Glu Thr Asp Thr Leu Leu Leu Trp Val Leu Leu Leu Trp Val Pro |
| (SEQ ID NO: 26) | Gly Ser Thr Gly |
| Ub (A) acceptor | Met Gln Ile Phe Val Arg Thr Leu Thr Asp Arg Thr Ile Thr Leu Glu |
| ubiquitin | Val Glu Pro Ser Asp Thr Ile Glu Asn Val Arg Ala Arg Ile Gln Asp Arg |
| (SEQ ID NO: 27) | Glu Gly Ile Pro Pro Asp Gln Gln Arg Leu Ile Phe Ala Gly Arg Gln |
| | Leu Glu Asp Gly Arg Thr Leu Ser Asp Tyr Asn |
| | IleGlnLysGluSerThrLeuHisLeuValLeuArgProArgValValAsp |
| Hinge (IgG1) (SEQ | Glu Pro Lys Ser Cys Asp Lys Thr His Thr Cys Pro |
| ID NO: 28) | |
| Fc (IgG1) (SEQ ID | Pro Cys Pro Ala Pro Glu Leu Leu Gly Gly Pro Ser Val Phe Leu Phe |
| NO: 32) | Pro Pro Lys Pro Lys Asp Thr Leu Met Ile Ser Arg Thr Pro Glu Val |
| | Thr Cys Val Val Val Asp Val Ser His Glu Asp Pro Glu Val Lys Phe |
| | Asn Trp Tyr Val Asp Gly Val Glu Val His Asn Ala Lys Thr Lys Pro |
| | Arg Glu Glu Gln Tyr Asn Ser Thr Tyr Arg Val Val Ser Val Leu Thr |
| | Val Leu His Gln Asp Trp Leu Asn Gly Lys Glu Tyr Lys Cys Lys Val |
| | Ser Asn Lys Ala Leu Pro Ala Pro Ile Glu Lys Thr Ile Ser Lys Ala Lys |
| | Gly Gln Pro Arg Glu Pro Gln Val Tyr Thr Leu Pro Pro Ser Arg Asp |
| | Glu Leu Thr Lys Asn Gln Val Ser Leu Thr Cys Leu Val Lys Gly Phe |
| | Tyr Pro Ser Asp Ile Ala Val Glu Trp Glu Ser Asn Gly Gln Pro Glu |
| | Asn Asn Tyr Lys Thr Thr Pro Pro Val Leu Asp Ser Asp Gly Ser Phe |
| | Phe Leu Tyr Ser Lys Leu Thr Val Asp Lys Ser Arg Trp Gln Gln Gly |
| | Asn Val Phe Ser Cys Ser Val Met His Glu Ala Leu His Asn His Tyr |
| | Thr Gln Lys Ser Leu Ser Leu Ser Pro Gly Lys |

The above Fc based acceptor protein recombinant expression vector was obtained.

Fc based acceptor protein plasmid DNA was put into DH5a competent cells, transformed by heat shock treatment at 42° C. for 1 minute, and plated on the LB solid medium containing 100 μg/mL ampicillin. The plated LB solid medium plate was stationary incubated at 37° C. for at least 16 hours to obtain colonies. A single colony was taken, inoculated into 5 mL of LB medium, and then incubated at 37° C. and 220 rpm for 16 hours. A part of the cultured solution was inoculated into LB medium containing ampicillin and then incubated at 37° C. and 220 rpm for 16 hours. The cultured solution was centrifuged at 3,500 rpm for 30 minutes to obtain an E. coli pellet, and then the P1, P2, and P3 solutions of the DNA extraction kit (QIAGEN) were added to break the cell wall, and the proteins were separated to obtain a DNA suspension. A plasmid DNA pellet was obtained from the DNA suspension obtained using a purification column of the DNA extraction kit (QIAGEN) and dried naturally. Water for cell culture (Sigma Aldrich) was added to the dried DNA pellet, dissolved, and then filtered by a 0.22 μm filter. The final extracted plasmid DNA was used for protein expression after measuring the DNA concentration and purity using a nano drop instrument (IMPLEN).

### Example 15: Expression and Confirmation Identification of Fc Based Acceptor Protein (Acceptor Protein)

### Expression of Fc Based Acceptor Protein

Expi293F human cells are derived from the human embryonic kidney 293 cell line and have high transfection and high protein expression efficiency.

24 hours prior to the transfection process, Expi293F (Gibco) cells were inoculated at 3×10⁶ viable cells/mL, placed on an Orbital shaker in an 8% CO₂ incubator, and incubated for 24 hours at 37° C., 80% humidity or higher, 95 rpm (a shaking diameter of 50-mm) conditions. The cells were counted to determine the cell viability and the cell number, diluted with the Expi293 expression medium (Gibco) to a final concentration of 3×10⁶ viable cells/mL and a total volume of 200 mL, and inoculated into a 1 L Erlenmeyer flask.

200 μg of an Fc based acceptor protein recombinant expression vector DNA was diluted in 12 mL of the Opti-MEM I Reduced Serum Media (Gibco) medium, and 640 μL of the ExpiFectamine™ 293 Reagent (Gibco) was diluted in 11.2 mL of the Opti-MEM I Reduced Serum Media (Gibco). Then, the reaction was carried out at ambient temperature for 5 minutes. The solution containing the ExpiFectamine™ 293 Reagent was put into a solution containing an Fc based acceptor protein recombinant expression vector DNA, mixed, and reacted at ambient temperature for 12 minutes. Transfection was carried out by slowly dispensing it into a 1 L flask inoculated to 3×10⁶ viable cells/mL. It was placed on an Orbital shaker in an 8% CO₂ incubator and incubated for 18 hours at 37° C., 80% humidity or higher, 95 rpm (a shaking diameter of 50-mm) conditions. After 18 hours, 1.2 mL of Enhancer 1 (Gibco) and 12 mL of Enhancer 2 (Gibco) were added, respectively, placed on an Orbital shaker in an 8% CO₂ incubator, and incubated for 7 days at 37° C., 80% humidity or higher, 95 rpm (a shaking diameter of 50-mm) conditions.

### Identification of Expression of Fc Based Acceptor Protein

The cultured solution obtained above was centrifuged for at least 30 minutes at a 3,500 rpm condition to obtain only a cultured solution of Fc based acceptor protein expression except for the cell pellet. The obtained cultured solution was filtered through a filter to remove impurities. In order to determine the expression level of an Fc based acceptor protein from the cultured solution, 80 μL was taken, and 20 μL of 5× non-reducing sample loading dye was added thereto, mixed, and let stood at 95° C. for 10 minutes.

In order to qualitatively analyze the expression level, 80 μL of bovine serum albumin that was diluted to 31.25, 62.5, 125, 250, 500, 750, and 1,000 mg/mL was taken, and 20 μL of 5× non-reducing sample loading dye was added, mixed, and let stood at 95° C. for 10 minutes. Each sample and a marker protein for size check were loaded on a 10% Tris-glycine gel, and the proteins were separated at 80 volts (V) for about 20 minutes and at 120 volts (V) for 90 minutes. When the gel running was completed, staining was performed with Coomassie brilliant blue R while gently shaking, and the staining reagent was removed from the stained gel using a buffer containing 10% acetic acid while gently shaking. The decolorized gel was obtained as an image file, and the concentration of the Fc based acceptor protein compared to the amount of the bovine serum albumin protein band was analyzed qualitatively and quantified through the Image J program, and the results are shown in FIG. 32.

As shown in FIG. 32, it was confirmed that an Fc based acceptor protein having a size of 100 kDa was formed, and it was confirmed that 264.1 mg/mL of the protein was formed.

### Confirmation of Target Specificity of Fc Based Acceptor Protein

Western blotting was performed to confirm the target specific expression of an Fc based acceptor protein. 80 μL of the expressed cultured solution was taken, and 20 μL of 5× non-reducing sample loading dye was added, mixed, and let stood at 95° C. for 10 minutes. The prepared sample and a marker protein for size check were loaded on a 10% Tris-glycine gel, and the proteins were separated at 80 volts (V) for about 20 minutes and at 120 volts (V) for 90 minutes. The gel transferred the protein on the polyvinylidene fluoride membrane (PVDF membrane) for about 2 hours at 0.3 amps (A) for electrophoresis. Blocking was performed on the membrane to remove non-specific reactions while gently shaking for 1 hour in 1×PBST (Phosphate Buffer Saline with Tween 20) containing 5% skim milk. Goat anti-rabbit IgG (H+L), which specifically binds to a human IgG Fc antibody, and HRP were used to confirm the Fc based acceptor protein specific expression. The results are shown in FIG. 3. A negative control is a sample that does not contain plasmid DNA.

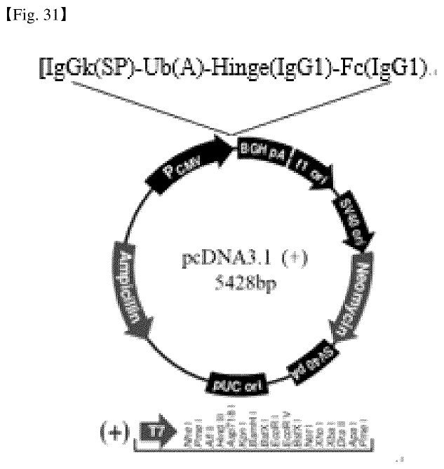

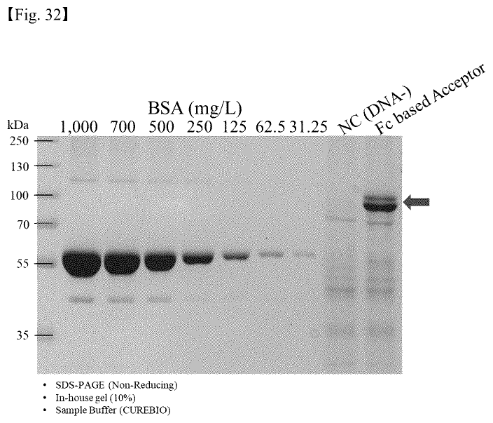

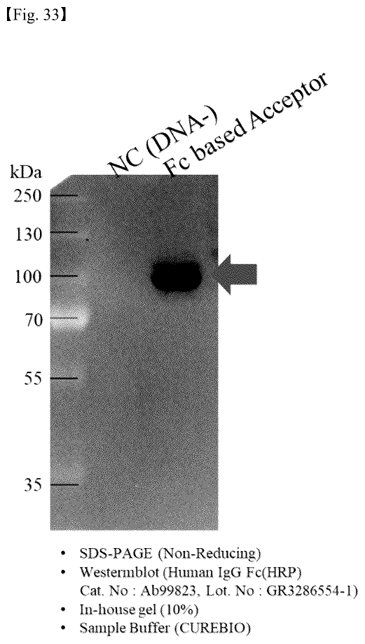

As shown in FIG. 33, the expression of an Fc based acceptor protein that specifically binds to an IgG Fc antibody and has a size of 100 kDa was confirmed.

### Purification of Fc Based Acceptor Protein

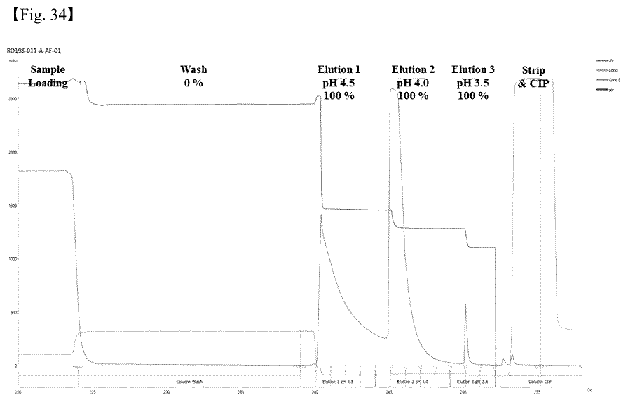

The cultured solution of the Fc based acceptor protein expressed above was loaded on a MabSelect Prism A (Cytiva) column equilibrated with an equilibrium buffer (20 mM sodium phosphate, 150 mM NaCl, pH 7.4). An equilibration buffer used to remove impurities that were not bound to the column, and Elution buffer 1 (50 mM sodium acetate at pH 4.5) and Elution buffer 2 (50 mM sodium acetate at pH 4.0) were used as a step elution to recover the acceptor protein. 1 M Tris was added to the recovered acceptor protein to allow the recovered protein to be at a pH of 7.5. The acceptor protein recovered after pH titration was subjected to dialysis with a 25 mM Tris, pH 7.5 buffer, followed by ultrafiltration. The results of the acceptor protein purification through SDS-PAGE were confirmed using a 10% in-house gel. The results are shown in FIGS. 34 and 35.

### Example 16: Expression and Purification of Ubiquitin-IL-1RA Protein (Donor Protein)

### Expression of Ubiquitin-IL-1RA Protein

A ubiquitin-biomolecule protein in which a biomolecule is directly bound to the C-terminus of the donor ubiquitin was prepared as follows.

The gene sequence encoding the ubiquitin-biomolecule protein was transformed into a His-SUMO tagged pET21a vector, and then 0.5 μL of a plasmid into which the gene was inserted was put into an E-tube containing 50 μL of E. coli BL21 (DE3), a competent cell. Thereafter, it was mixed by tapping and then let stood in ice for 20 minutes. In order to apply a heat shock, the E-tube was let stood in a water bath at 42° C. for 50 seconds and then let stood in ice for 5 minutes. Thereafter, 300 μL of fresh LB medium was added to the E-tube and incubated in a 37° C. shaking incubator for 1 hour to complete transformation. The transformed cells were spread on an LB plate containing ampicillin at a concentration of 100 mg/ml in BSC in a 1/1000 ratio and incubated overnight in a 37° C. stationary incubator. Thereafter, the resulting single colony was taken and inoculated into 100 mL of TB medium containing ampicillin at a concentration of 100 mg/ml in a 1/1000 ratio, and then the seed was cultured at 37° C. and 220 rpm for 6 hours.

In the case of the ubiquitin-IL-1RA protein (Donor, D-192) containing IL-1RA as a biomolecule, the seed cultured solution was inoculated into 3 L of In-house TB medium containing in a 1/1000 ratio ampicillin at a concentration of 100 mg/ml in a 1:100 ratio, and main culture was carried out. Incubation was carried out at 37° C., dissolved oxygen 40% for 4 hours using the Biocanvas fermentor (Centrion), and induction of 1 M IPTG stock at a final concentration of 200 μM was carried out. In order to adjust the amount of dissolved oxygen in the cultured solution to 40%, the RPM of the impeller was automatically adjusted to 300 to 700 rpm during the incubation time. After induction, incubation was continued for another 14 hours and the incubation was terminated. The incubated solution was centrifuged at 7000 g for 30 minutes to obtain E. coli wet cells.

The remaining nutrients and optical density were measured after completion of incubation using Cedex BIO Analyzer (Roche). The results are shown in Table 5 below.

TABLE 5

| pH | Acetate | Glucose | Glycerol | Mg²⁺ | P | Optical |
|---|---|---|---|---|---|---|
| Meter | (mg/L) | (mg/L) | (mg/L) | (mg/L) | (mg/L) | density |
| 7.37 | 193.9 | 0.0 | 1190.7 | 56.2 | 6826.9 | 36.7 |

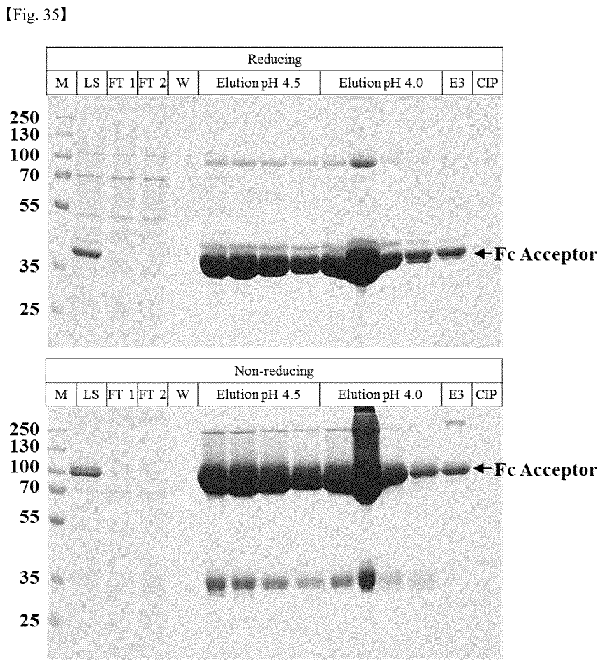

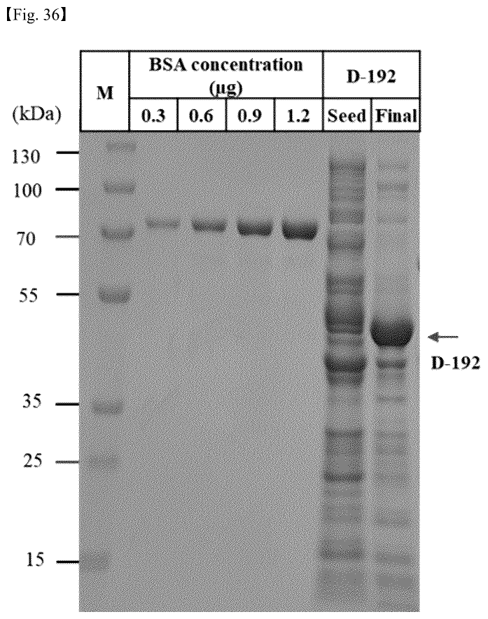

The ubiquitin-IL-1RA was identified through SDS-PAGE analysis method. The results are shown in FIG. 36. In addition, the concentration of the ubiquitin-IL-1RA protein was measured, and the result was analyzed to be 963.32 mg/L.

### Purification of Ubiquitin-IL-1RA Protein

The ubiquitin-IL-1RA protein was purified by the following process (FIG. 37).

### (A) Lysis/Sonication

Resuspension of wet cells obtained by incubation was carried out using a lysis buffer (20 mM sodium phosphate, pH 7.0). It was carried out by adding 9 mL of a lysis buffer per 1 g of wet cells. Lysis samples were placed on ice, and sonication was carried out for 20 minutes under conditions of Pules on/off=3 sec/5 sec, 45% amplitude. The lysate was centrifuged at 14,000 rpm for 30 minutes to obtain only supernatant.

### (B) Capture Purification

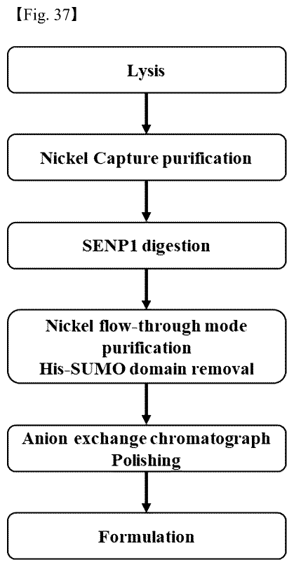

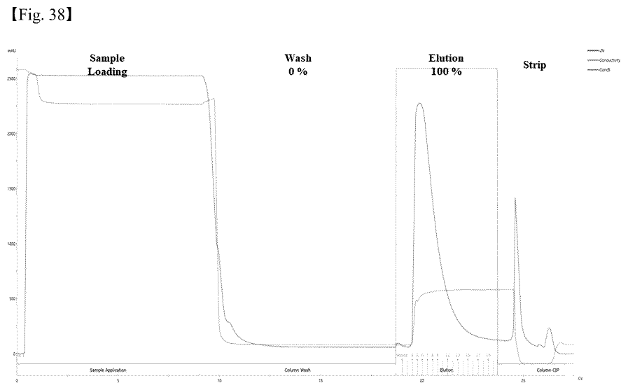

The lysate was loaded on a Ni-sepharose resin (Cytiva). After the sample loading was completed, it was sufficiently washed using a washing buffer (20 mM sodium phosphate, pH 7.0, 0.02 M imidazole) to remove the non-specific protein. Thereafter, the Hig-SUMO tagged ubiquitin-IL-1RA protein was recovered using an elution buffer (20 mM sodium phosphate, pH 7.0, 0.2 M imidazole). The recovered His-SUMO tagged ubiquitin-IL-1RA protein was subjected to dialysis with a 20 mM sodium phosphate, pH 7.0 buffer to remove imidazole. Purification of the donor protein through the Ni column was confirmed by SDS-PAGE. The results are shown in FIGS. 38 and 39.

### (C) SENP1 Enzyme Digestion

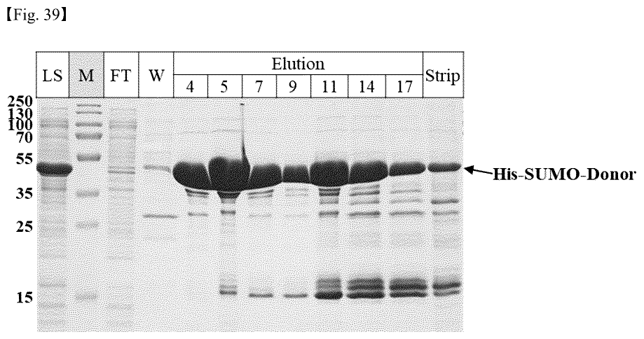

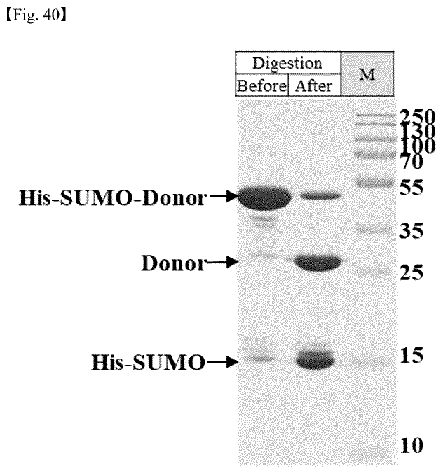

The His-SUMO tagged ubiquitin-IL-1RA protein and SENP1 were subjected to SENP1 enzyme digestion at a ratio of 100 mg of ubiquitin-IL-1RA protein: 1 mg of SENP1. The concentration of the recovered protein was quantified by Ni-purification, and the corresponding recombinant SNEP1 was mixed based on the amount of the His-SUMO tagged ubiquitin-IL-1RA protein. The reaction mixture was let stood at ambient temperature (15 to 25° C.) for 1 hour. The SENP1 enzyme digestion was confirmed by SDS-PAGE. The results are shown in FIG. 40.

### (D) Removal of His-SUMO

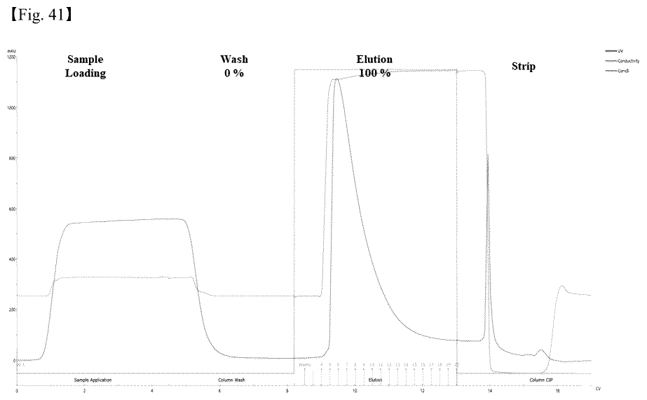

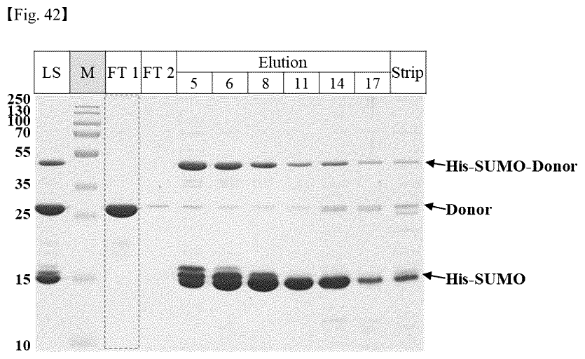

The reaction mixture was loaded on a Ni-sepharose resin (Cytiva) equilibrated with an equilibrium buffer (20 mM sodium phosphate, pH 7.0, 0.02 M imidazole). Sample loading was performed, and the ubiquitin-IL-1RA protein in which His-SUMO tag was cleaved was allowed to flow through. After the sample loading was completed, the remaining ubiquitin-IL-IRA protein was recovered using an equilibrium buffer (20 mM sodium phosphate, pH 7.0, 0.02 M imidazole), and the recovered ubiquitin-IL-1RA protein was subjected to dialysis with a 20 mM sodium phosphate, pH 7.0 buffer to remove imidazole. The process in which His-SUMO is removed was confirmed by SDS-PAGE. The results are shown in FIGS. 41 and 42.

### (E) Purification

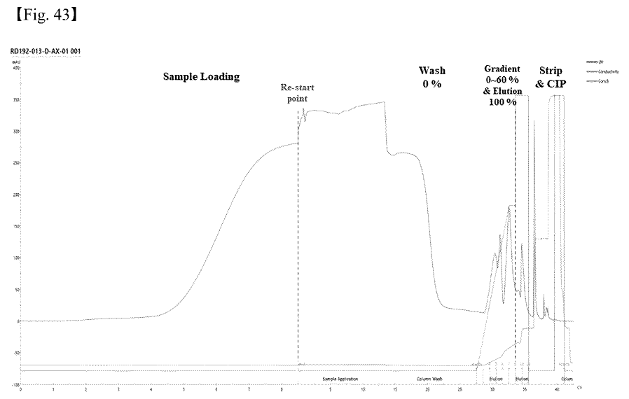

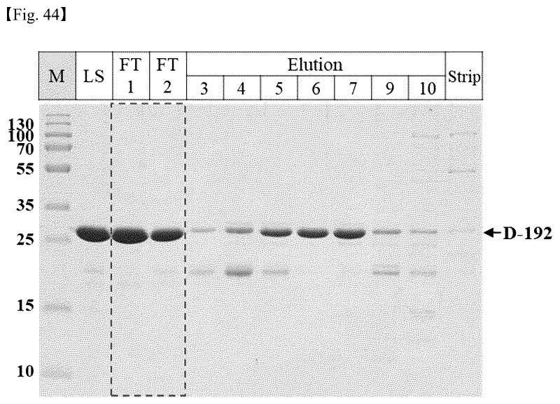

The ubiquitin-IL-1RA protein recovered in the previous step was loaded on an anion exchange resin column equilibrated with an equilibrium buffer (20 mM sodium phosphate, pH 7.0 buffer). The ubiquitin-IL-1RA protein was allowed to flow through. The recovered ubiquitin-IL-1RA protein was subjected to ultrafiltration to a final concentration of 10 mg/mL. The results of the anion exchange resin process for polishing are shown in FIGS. 43 and 44.

### Example 17: Conjugation

### Performance and Yield of Conjugation

Conjugation was performed using the acceptor protein produced in Example 18 and the donor protein produced in Example 19. The molar ratio of the acceptor and donor proteins was 1:3. At this time, the acceptor protein can be set to 10 μM to 50 μM. In addition, E1, E2, E3, and ATP were added to the UniStac mixture to initiate the reaction. The reaction was carried out at 25° C. for 16 hours in a stationary state.

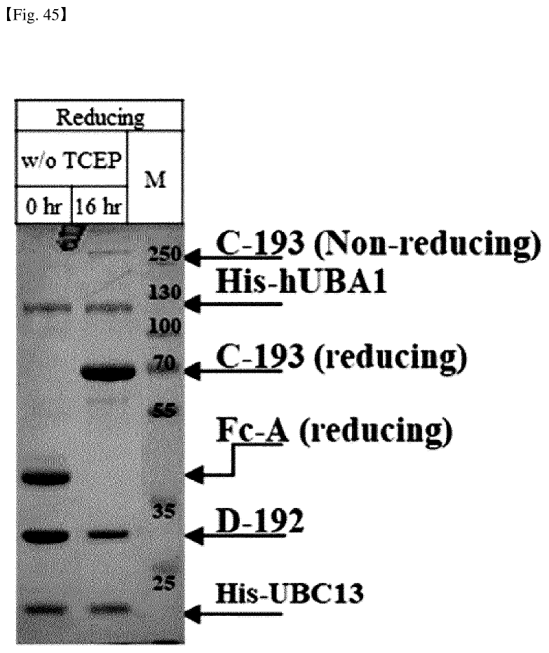

For the resulting product of conjugation reaction (C-193), the degree of conjugation was qualitatively analyzed by loading 1.12 μg of the acceptor on a 4 to 12% gradient SDS-PAGE, and the results are shown in FIG. 45.

In addition, for the resulting product of the reaction, the degree of conjugation was quantitatively analyzed using μCE-SDS analysis method. Sample pretreatment was performed using the HT Protein Express Reagent Kit, and then analysis was performed using the Protein Express Assay Labchip, and the yield analysis results are shown in Table 6 below and FIG. 46.

TABLE 6

| | | | Conjugation yield |
|---|---|---|---|
| Reaction step | Sample | Correlative Area | (%) |
| Pre-reaction | acceptor protein | 6092.70 | 97.21 |
| | conjugate (C-193) | 0.00 | |
| Post-reaction | acceptor protein | 169.70 | |
| | conjugate (C-193) | 11150.76 | |

$$UniStac conjugaiton yield ( \% ) = 100 - ( \frac{After Acceptor Corr \cdot Area}{Before Acceptor Corr \cdot Area} \times 100 ) \qquad [ Equation 1 ]$$

The conjugation yield in Table 6 above was calculated using Equation 1 above, and the UniStac conjugation yield ws 97.21%, indicating a very high acceptor specific value.

### Purification of UniStac Conjugation

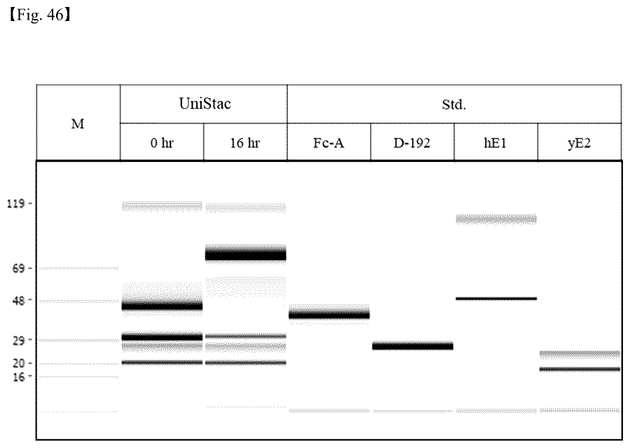

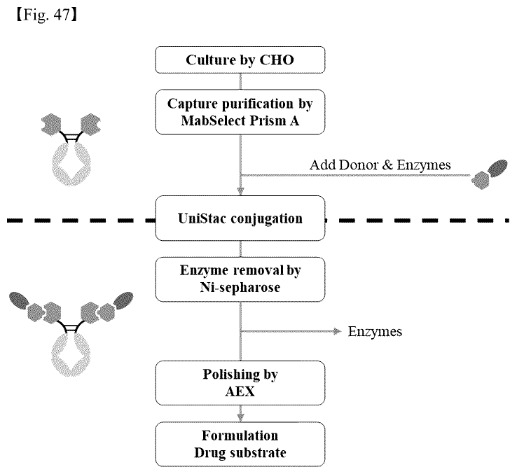

In order to recover only the conjugation (C-193) sample, purification was carried out using the following process (FIG. 47).

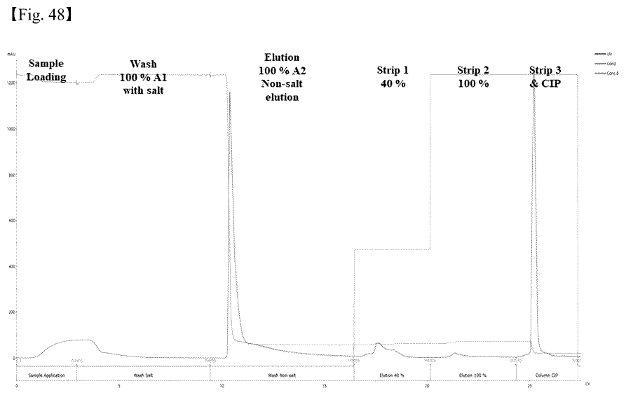

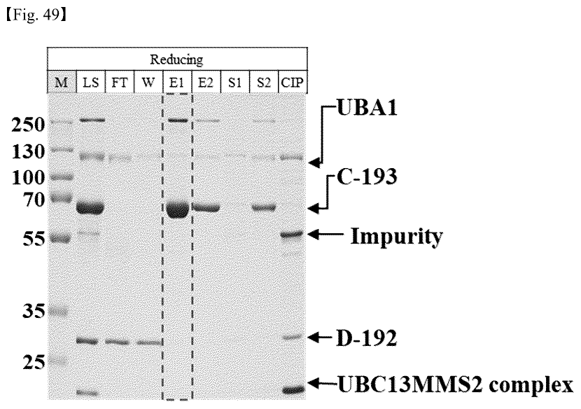

For the resulting product of the reaction, a Ni-sepharose resin equilibrated with an equilibrium buffer (25 mM Tris, pH 8.0, 0.5 M NaCl) was prepared. A small amount of 5 M NaCl was added to the loading sample to adjust the conductivity to 50 mS/cm. After loading the sample prepared with the corresponding conductivity into the prepared column was completed, an equilibrium buffer (25 mM Tris, pH 8.0, 0.5 M NaCl) was used to remove impurities. Thereafter, an elution buffer (25 mM Tris, pH 8.0) was used to recover the conjugation (C-193). The recovered conjugation (C-193) was subjected to dialysis with a pH 7.0 sodium phosphate buffer without salt to remove salt and imidazole. The Ni purification was confirmed through chromatography, and the results are shown in FIGS. 48 and 49.

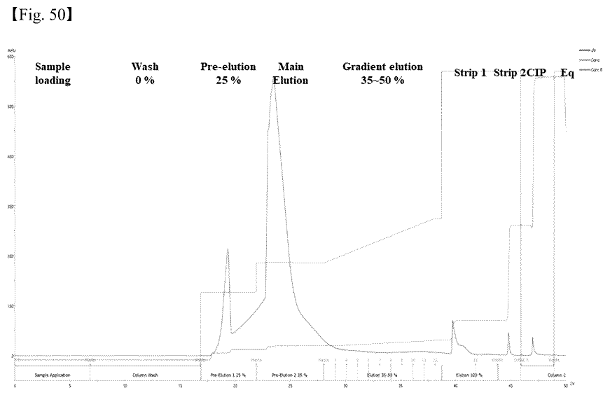

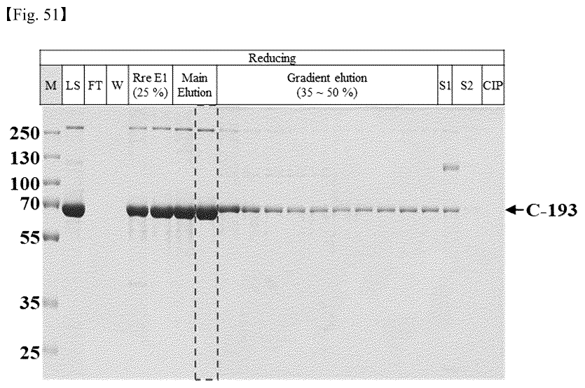

The conjugation was loaded on an anion exchange resin (anion exchange chromatography) column equilibrated with an equilibrium buffer (25 mM sodium phosphate, pH 7.0 buffer). An elution buffer (25 mM sodium phosphate, pH 7.0, 250 mM NaCl) was used to recover the conjugation, and the results are shown in FIGS. 50 and 51.

### Final UniStac

The conjugation (C-193) was subjected to dialysis with a formulation buffer (4.6 mM histidine, 5.7 mM Tris, pH 7.5, 10 mM arginine, 0.1 g/mL trehalose) to perform the formulation. The final UniStac product was prepared by dilution to 1.1 mg/mL and 0.5 mg/mL. The produced samples were stored in a −70° C. deep freezer.

### Example 18: Physicochemical Analysis of Final UniStac Polymer

In order to measure the purity of the final UniStac polymer (Drug Substrate, C-193), SDS-PAGE (reducing and native conditions), μCE-SDS and SEC-HPLC analysis were carried out.

### SDS-PAGE Analysis

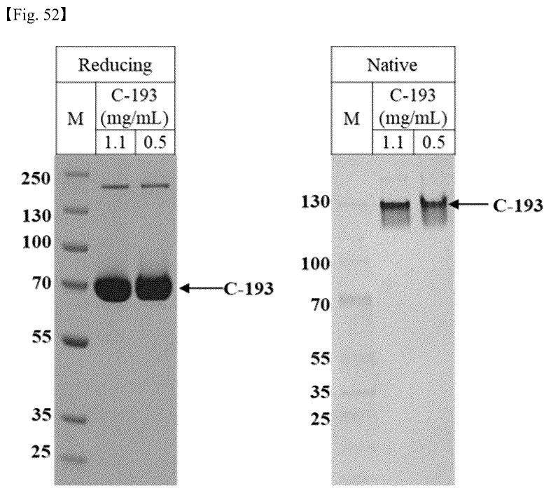

In order to analyze the final conjugate product (DS, C-193) under reducing conditions, reducing SDS-PAGE analysis was carried out using 4-12% Bis-Tris Plus Gel and MES SDS Running Buffer. 3 μg of the sample was loaded into each of the prepared PAGE, and the results are shown in FIG. 52.

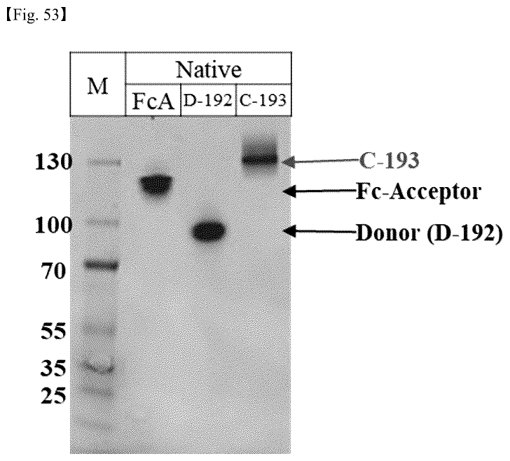

In order to analyze the final conjugate product (C-193 DS) under native conditions, native-PAGE analysis was carried out using 4-15% T/G-PAG-BC non-SDS and Tris-glycine native Running Buffer. 4.5 μg of the sample was loaded into each of the prepared PAGE, and the results are shown in FIG. 53.

### μCE-SDS Analysis

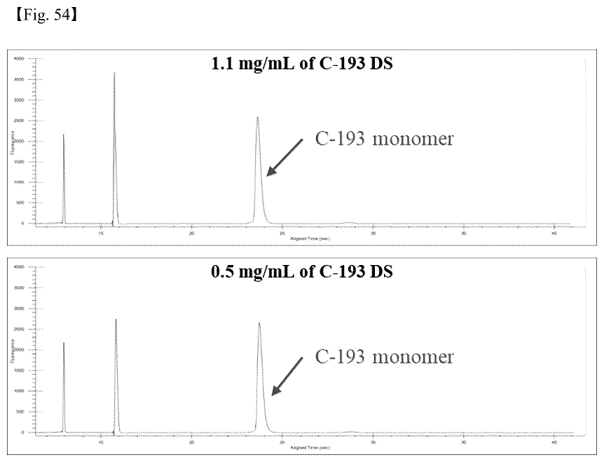

The degree of fragment inclusion in the C-193 DS sample was analyzed using μCE-SDS (Perkin Elmer Labchip GX II Touch.) analysis method. The sample pretreatment was carried out using a HT Protein Express Reagent Kit (Perkin Elmer), and then the analysis was carried out using a Protein Express Assay Labchip (Perkin Elmer). The results are shown in Table 7 below and FIG. 54.

TABLE 7

| | Migration time | | Purity |
|---|---|---|---|
| Concentration of UniStac sample | (sec) | Corr. Area | (%) |
| 1.1 mg/mL | 23.62 | 3527.21 | 98.12 |
| 0.5 mg/mL | 24.56 | 3616.85 | 98.69 |

$$Purity ( \% ) = ( \frac{C - 193 Corr \cdot Area}{Total Corr \cdot Area} \times 100 ) \qquad [ Equation 2 ]$$

The purity of the monomer was calculated using Equation 2, and the results are shown in Table 7, confirming that the purity of the monomer was high as 98% or more.

### SEC-HPLC Analysis

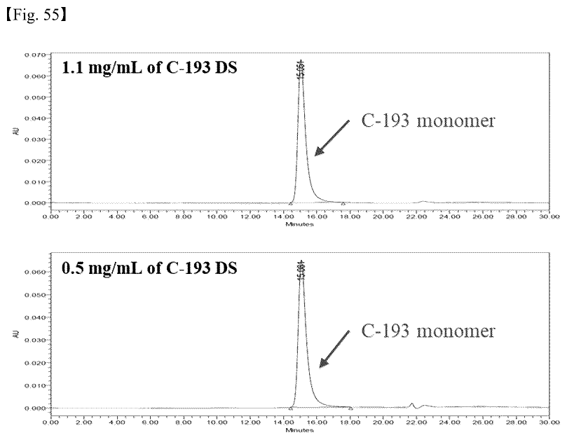

The degree of high molecular weight inclusion in the C-193 DS sample was analyzed using SEC-HPLC column and Alliance e2695 XC HPLC instrument. About 30 μg of the C-193 DS sample was injected into each of the prepared column to perform analysis, and the results are shown in Table 8 below and FIG. 55.

TABLE 8

| | Retention Time | | |
|---|---|---|---|
| Concentration of sample | (min) | Area | Purity % |
| 1.1 mg/mL | 15.051 | 2234084 | 100.00 |
| 0.5 mg/mL | 15.081 | 2264850 | 100.00 |

The purity of the monomer was calculated using Equation 2 above, and the results are shown in Table 8 above, confirming that the purity of the monomer was very high as 100%.

### Example 19: Comparison of Pharmacokinetics of Fusion Proteins (C-192, C-193 and D-192)

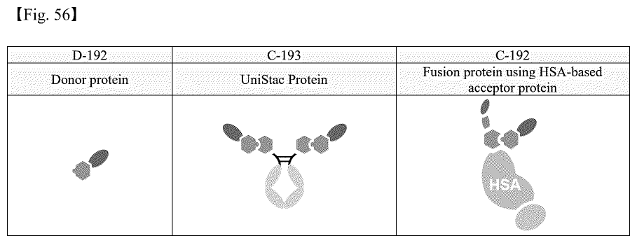

The donor protein (D-192), the UniStac protein (C-193), and the fusion protein (C-192) using an acceptor protein using a human-serum albumin as a carrier were prepared. The structures of the three samples are shown in FIG. 56 below.

9-week-old male ICR mice were subcutaneously administered once with the three samples, and then the blood was collected for each time period, and a pharmacokinetics measurement test was carried out to analyze the blood drug concentration and calculate the pharmacokinetic parameters.

8-week-old male ICR mice purchased from Orient BIO, Korea were quarantined and acclimatized for 7 days. After quarantine and acclimatization, body weights were ranked for all animals, and group separation (n=2 per blood collection time) was performed so that the average body weight of each group was uniformly distributed. Thereafter, the C-192 test substance at 10 mg/kg, the C-193 test substance at 1 mg/kg, and the D-192 test substance at 1 mg/kg dose were subcutaneously administered once to each mouse. Blood collection time points were before administration, 2, 6, 8, 10, 16, 32, 24, 48, 72 and 96 hours after administration of C-192, 0.5, 1, 2, 4, 6, 8, 10, 16, 24, 32, 40, 48, 56 and 72 hours after administration of C-193, and 1, 2, 3, 4, 5 and 6 hours after administration of D-192, respectively. The serum was separated from the collected blood by centrifugation and stored at −70=10° C. for analysis of blood drug concentration.

In order to measure the blood drug concentration of the test substance, the human IL-IRA ELISA kit (Abcam, UK) having a specific reactivity to IL-1RA was used. First, 50 μL of standard material and serum for each time period were dispensed into a 96 well plate, and then 50 μL of the antibody cocktail provided by the human IL-IRA ELISA kit was dispensed into each well, and the reaction was carried out in a 25° C. mixing device (Thermo Micromixer) at 400 rpm for 1 hour. The solution in the plate well was discarded and shaken so as not to leave any residue. 300 μL of the washing solution was dispensed into each well, and then the procedure of discarding and shaking was repeated three times. 100 μL of the color former was dispensed into each well, and then the reaction was carried out in a 25° C. mixing device at 400 rpm 10 minutes.

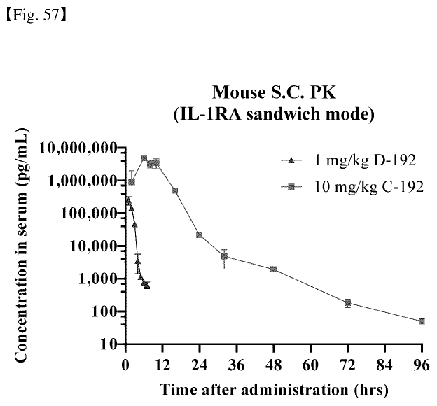

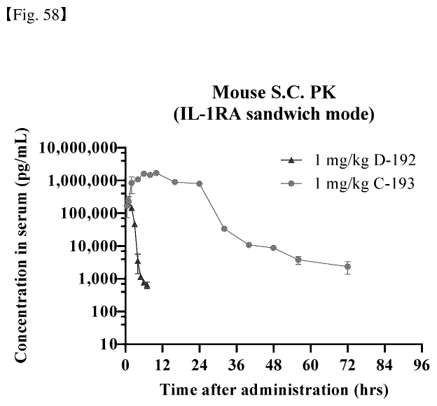

Finally, 100 μL of the stop solution was dispensed into each well, and then absorbance was measured at 450 nm using an absorbance measuring instrument (Multi-Mode Microplate Reader). The concentration of the drug in the serum for each time period calculated relative to the standard material was used to calculate the pharmacokinetic parameters. FIGS. 57 and 58 show graphs of blood drug concentrations over time after subcutaneous administration of the fusion proteins (C-192, C-193 and D-192) in mice.

In addition, the pharmacokinetic parameters were calculated based on the experimental results, and the results are shown in Table 9 below.

TABLE 9

| | C-192 | C-193 | D-192 |
|---|---|---|---|
| PK parameter | 10 mg/kg | 1 mg/kg | 1 mg/kg |
| Tₘₐₓ (hr) | 6 | 10 | 1 |
| Cₘₐₓ (pg/mL) | 4,853,634 | 1,696,762 | 250,240 |
| AUC (inf) (pg*hr/mL) | 37,378,687 | 28,185,225 | 426,747 |
| Half life (hr) | 9.3 | 13.7 | 0.4 |

As shown in Table 9 above, when the three fusion proteins were compared and evaluated based on the half-life indicating the in vivo stability of the drug, it was confirmed that the half-life of the fusion protein (C-192) using a human serum albumin as a carrier was increased by about 23 times from 0.4 hours to 9.3 hours compared to the acceptor protein (D-192).

In addition, it was confirmed that the half-life of the fusion protein (C-193) using Fc as a carrier was increased by about 33 times from 0.4 hours to 13.7 hours compared to the acceptor protein (D-192).

In the end, the fusion protein in which albumin and Fc carrier are fused has excellent pharmacokinetic properties by increasing half-life, AUC, Tmax, and Cmax, and can be utilized as an excellent drug that can be applied to a desired site with a small dose.

1. A multifunctional multispecific multimeric biomolecule polymer, comprising a polyubiquitin scaffold which is formed by covalently bonding two or more ubiquitins, and 2 to 10 biomolecules comprising binding moieties, each specific for different binding sites, wherein the biomolecule comprises active sites that specifically bind to other biomolecules, small molecule chemical compounds or nanoparticles, and is directly bound to the N-terminus, the C-terminus, or both the N-terminus and the C-terminus of the ubiquitin or is bound by a linker, and a carrier that prolongs the in vivo stability and duration of the biomolecule is directly bound to the N-terminus, the C-terminus, or both the N-terminus and the C-terminus of the ubiquitin or is bound by a linker.

2. The multifunctional multispecific multimeric biomolecule polymer according to claim 1, wherein the linker is a combination of 1 to 30 repeats of GGGGS (SEQ ID NO: 30) or EAAAK (SEQ ID NO:31).

3. The multifunctional multispecific multimeric biomolecule polymer according to claim 1, wherein the biomolecule bound to the N-terminus of the ubiquitin is the distal end of the multimeric biomolecule polymer.

4. The multifunctional multispecific multimeric biomolecule polymer according to claim 1, wherein the biomolecule bound to the C-terminus, the N-terminus, or both the C-terminus and the N-terminus of the ubiquitin is the proximal end of the multimeric biomolecule polymer.

5. The multifunctional multispecific multimeric biomolecule polymer according to claim 1, wherein the carrier is one or more selected from the group consisting of albumin, antibody fragment, Fc domain, transferrin, XTEN (genetic fusion of non-exact repeat peptide sequence), CTP (carboxy-terminal peptide), PAS (proline-alanine-serine polymer), ELK (elastin-like peptide), HAP (homo-amino acid polymer), GLK (gelatin-like protein), PEG (polyethylene glycol), and fatty acid.

6. The multifunctional multispecific multimeric biomolecule polymer according to claim 1, wherein the polyubiquitin scaffold is formed by covalently bonding a donor ubiquitin in which one or more lysines of the ubiquitin are substituted with other amino acids including arginine or alanine, and an acceptor ubiquitin in which the 6th, 11th, 27th, 29th, 33rd, 48th, or 63rd lysine from the N-terminus is substituted with other amino acids including arginine or alanine.

7. The multifunctional multispecific multimeric biomolecule polymer according to claim 1, wherein the 73rd leucine from the N-terminus of the ubiquitin is substituted with other amino acids including proline.

8. The multifunctional multispecific multimeric biomolecule polymer according to claim 1, wherein the biomolecule is selected from the group consisting of insulin, insulin analogue, glucagon, glucagon-like peptides, GLP-1 and glucagon dual agonist, GLP-1 and GIP dual agonist, GLP-1 and glucagon and GIP triple agonist, exendin-4, exendin-4 analogue, insulin secreting peptide and an analogue thereof, human growth hormone, growth hormone releasing hormone (GHRH), growth hormone releasing peptide, granulocyte colony stimulating factor (G-CSF), anti-obesity peptide, G-protein-coupled receptor, leptin, GIP (gastric inhibitory polypeptide), interleukins, interleukin receptors, interleukin binding proteins, interferons, interferon receptors, cytokine binding proteins, macrophage activator, macrophage peptide, B cell factor, T cell factor, suppressive factor of allergy, cell necrosis glycoprotein, immunotoxin, lymphotoxin, tumor necrosis factor (TNF), tumor inhibitory factor, metastasis growth factor, alpha-1 antitrypsin, albumin, α-lactalbumin, apolipoprotein-E, erythropoietin (EPO), high glycosylated erythropoietin, angiopoietins, hemoglobin, thrombin, thrombin receptor activating peptide, thrombomodulin, blood factors VII, VIIa, VIII, IX, and XIII, plasminogen activator, fibrin-binding peptide, urokinase, streptokinase, hirudin, protein C, C-reactive protein, renin inhibitor, collagenase inhibitor, superoxide dismutase, platelet derived growth factor, epithelial growth factor, epidermal growth factor, angiostatin, angiotensin, bone morphogenetic growth factor, bone morphogenetic protein, calcitonin, atriopeptin, cartilage inducing factor, elcatonin, connective tissue activator, tissue factor pathway inhibitor, follicle stimulating hormone (FSH), luteinizing hormone (LH), luteinizing hormone releasing hormone (LHRH), nerve growth factors, parathyroid hormone (PTH), relaxin, secretin, somatomedin, adrenal cortical hormone, cholecystokinin, pancreatic polypeptide, gastrin releasing peptide, corticotropin releasing factor, thyroid stimulating hormone (TSH), autotaxin, lactoferrin, myostatin, receptor, receptor antagonist, fibroblast growth factor, adiponectin, interleukin receptor antagonist, cell surface antigen, virus derived vaccine antigen, monoclonal antibody, polyclonal antibody and antibody fragments.

9. A method for preparing a multifunctional multispecific multimeric biomolecule polymer, in which a polyubiquitin scaffold, two or more biomolecules comprising binding moieties, each specific for different binding sites, and a carrier that prolongs the in vivo duration are directly bound to the N-terminus or the C-terminus of the ubiquitin or are bound by a linker, wherein the method comprises
(i) recombinantly expressing a biomolecule to which a ubiquitin C-terminal tag is fused or bound by a linker from a host cell including a procaryotic cell or a eukaryotic cell, and
(ii) adding E1, E2 and E3 enzymes, or E1 and E2 enzymes for ubiquitination to the cell lysates or purified products of the host cell and reacting them,
wherein the polyubiquitin scaffold is formed by covalently bonding two or more ubiquitins, and
the biomolecule is composed of 2 to 10 biomolecules, has active sites that specifically bind to other biomolecules, small molecule chemical compounds or nanoparticles, and is bound to the N-terminus, the C-terminus, or both the N-terminus and the C-terminus of the ubiquitin by a linker.

10. The method according to claim 9, wherein the E2 enzyme binds to the 6th, 11th, 27th, 29th, 33rd, 48th or 63rd lysine from the N-terminus of the ubiquitin.

11. The method according to claim 9, wherein the E2 enzyme is an E2-25K ubiquitin conjugating enzyme.

12. The method according to claim 9, wherein the E2 enzyme is Ucb13-MMS2, a ubiquitin conjugating enzyme complex.

13. The method according to claim 9, wherein the ubiquitin C-terminal tag is one in which two or more ubiquitins are repeatedly linked in a head-to-tail form or in a branched form (branched type or iso-peptide branch type form).

14. The method according to claim 13, wherein the ubiquitin linked in a head-to-tail form or in a branched form is one in which the 75th and 76th glycines from the N-terminus are substituted with other amino acids including valine.


SEQUENCE LISTING (32 sequences)

SEQ ID NO: 1 - protein, 76 aa, Artificial Sequence [sequence omitted]

SEQ ID NO: 2 - protein, 506 aa, Artificial Sequence
  Xylose Reductase
MGHHHHHHSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGMPSIKLNSGYDMPAVGFGCWKVDVDTCSEQIYRAIKTGYRLFDGAEDYANEKLVGAGVKKAIDEGIVKREDLFLTSKLWNNYHHPDNVEKALNRTLSDLQVDYVDLFLIHFPVTFKFVPLEEKYPPGFYCGKGDNFDYEDVPILETWKALEKLVKAGKIRSIGVSNFPGALLLDLLRGATIKPSVLQVEHHPYLQQPRLIEFAQSRGIAVTAYSSFGPQSFVELNQGRALNTSPLFENETIKAIAAKHGKSPAQVLLRWSSQRGIAIIPKSNTVPHLLENKDVNSFDLDEQDFADIAKLDINLRFNDPWDWDKIPIFVGGHHHHHHMQIFVKTLTGKTITLEVEPSDTIENVKAKIQDKEGIPPDQQRLIFAGKQLEDGRTLSDYNIQKESTLHLVLRLRGG

SEQ ID NO: 3 - protein, 542 aa, Artificial Sequence
  Xylitol dehydrogenase
MGHHHHHHSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGMTANPSLVLNKIDDISFETYDAPEISEPTDVLVQVKKTGICGSDIHFYAHGRIGNFVLTKPMVLGHESAGTVVQVGKGVTSLKVGDNVAIEPGIPSRFSDEYKSGHYNLCPHMAFAATPNSKEGEPNPPGTLCKYFKSPEDFLVKLPDHVSLELGALVEPLSVGVHASKLGSVAFGDYVAVFGAGPVGLLAAAVAKTFGAKGVIVVDIFDNKLKMAKDIGAATHTFNSKTGGSEELIKAFGGNVPNVVLECTGAEPCIKLGVDAIAPGGRFVQVGNAAGPVSFPITVFAMKELTLFGSFRYGFNDYKTAVGIFDTNYQNGRENAPIDFEQLITHRYKFKDAIEAYDLVRAGKGAVKCLIDGPEMQIFVKTLTGKTITLEVEPSDTIENVKAKIQDKEGIPPDQQRLIFAGKQLEDGRTLSDYNIQKESTLHLVLRLRG

SEQ ID NO: 4 - protein, 407 aa, Artificial Sequence
  Oxaloacetate decarboxylase
MGHHHHHHSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGMYELGVVYRNIQRADRAAADGLAALGSATVHEAMGRVGLLKPYMRPIYAGKQVSGTAVTVLLQPGDNWMMHVAAEQIQPGDIVVAAVTAECTDGYFGDLLATSFQARGARALIIDAGVRDVKTLQEMDFPVWSKAISSKGTIKATLGSVNIPIVCAGMLVTPGDVIVADDDGVVCVPAARAVEVLAAAQKRESFEGEKRAKLASGVLGLDMYKMREPLEKAGLKYIDMQIFVKTLTGKTITLEVEPSDTIENVKAKIQDKEGIPPDQQRLIFAGKQLEDGRTLSDYNIQKESTLHLVLRLRGG

SEQ ID NO: 5 - protein, 430 aa, Artificial Sequence
  Triose-phosphate isomerase
MGHHHHHHSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGMRRVLVAGNWKMHKTPSEARVWFAELKRLLPPLQSEAAVLPAFPILPVAKEVLAETQVGYGAQDVSAHKEGAYTGEVSARMLSDLGCRYAIVGHSERRRYHGETDALVAEKAKRLLEEGITPILCVGEPLEVREKGEAVPYTLRQLRGSLEGVEPPGPEALVIAYEPVWAIGTGKNATPEDAEAMHQEIRKALSERYGEAFASRVRILYGGSVNPKNFADLLSMPNVDGGLVGGASLELESFLALLRIAGMQIFVKTLTGKTITLEVEPSDTIENVKAKIQDKEGIPPDQQRLIFAGKQLEDGRTLSDYNIQKESTLHLVLRLRGG

SEQ ID NO: 6 - protein, 496 aa, Artificial Sequence
  Aldolase
MGHHHHHHSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGMVTMPYVKNTKEILEKASKERYAIGAFNFNNMEFLQAILEAAEEEKAPVIVATSEGAIKYIGKGDIETGAKLAVEMVRTYAEKLSVPVALHLDHGRDFKVIMAAIKAGYSSVMIDASHLPFEENLRETKRIVEIAHAVGISVEAELGKLKGIEDNVVEKESVLVDPEEAKVFVKETEVDFLAPAIGTSHGAFKFKGEAQLDFERLKKVKEYTQIPLVLHGASMVPQDIVKLANEYGAELSGAKGVPEDMLKKAIELGINKINTDTDLRITFVAYLRKVLSEDKSQIDPRKIFKPVFEQVKEIVKERIRIFGSSGKAMQIFVKTLTGKTITLEVEPSDTIENVKAKIQDKEGIPPDQQRLIFAGKQLEDGRTLSDYNIQKESTLHLVLRLRGG

SEQ ID NO: 7 - protein, 437 aa, Artificial Sequence
  Fructose 1,6-bisphosphatase
MGHHHHHHSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGMLDRLDFSIKLLRKVGHLLMIHWGRVDNVEKKTGFKDIVTEIDREAQRMIVDEIRKFFPDENIMAEEGIFEKGDRLWIIDPIDGTINFVHGLPNFSISLAYVENGEVKLGVVHAPALNETLYAEEGSGAFFNGERIRVSENASLEECVGSTGSYVDFTGKFIERMEKRTRRIRILGSAALNAAYVGAGRVDFFVTWRINPWDIAAGLIIVKEAGGMVTDFSGKEANAFSKNFIFSNGLIHDEVVKVVNEVVEEIGGKMQIFVKTLTGKTITLEVEPSDTIENVKAKIQDKEGIPPDQQRLIFAGKQLEDGRTLSDYNIQKESTLHLVLRLRGG

SEQ ID NO: 8 - protein, 772 aa, Artificial Sequence
  Pyruvate oxidase
MGHHHHHHSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGMSDNKINIGLAVMKILESWGADTIYGIPSGTLSSLMDAMGEEENNVKFLQVKHEEVGAMAAVMQSKFGGNLGVTVGSGGPGASHLINGLYDAAMDNIPVVAILGSRPQRELNMDAFQELNQNPMYDHIAVYNRRVAYAEQLPKLVDEAARMAIAKRGVAVLEVPGDFAKVEIDNDQWYSSANSLRKYAPIAPAAQDIDAAVELLNNSKRPVIYAGIGTMGHGPAVQELARKIKAPVITTGKNFETFEWDFEALTGSTYRVGWKPANETILEADTVLFAGSNFPFSEVEGTFRNVDNFIQIDIDPAMLGKRHHADVAILGDAGLAIDEILNKVDAVEESAWWTANLKNIANWREYINMLETKEEGDLQFYQVYNAINNHADEDAIYSIDVGNSTQTSIRHLHMTPKNMWRTSPLFATMGIAIPGGLGAKNTYPDRQVWNIIGDGAFSMTYPDVVTNVRYNMPVINVVFSNTEYAFIKNKYEDTNKNLFGVDFTDVDYAKIAEAQGAKGFTVSRIEDMDRVMAEAVAANKAGHTVVIDCKITQDRPIPVETLKLDSKLYSEDEIKAYKERYEAANLVPFREYLEAEGLESKYIKMQIFVKTLTGKTITLEVEPSDTIENVKAKIQDKEGIPPDQQRLIFAGKQLEDGRTLSDYNIQKESTLHLVLRLRGG

SEQ ID NO: 9 - protein, 1024 aa, Artificial Sequence
  Yeast UBE1
MSSNNSGLSAAGEIDESLYSRQLYVLGKEAMLKMQTSNVLILGLKGLGVEIAKNVVLAGVKSMTVFDPEPVQLADLSTQFFLTEKDIGQKRGDVTRAKLAELNAYVPVNVLDSLDDVTQLSQFQVVVATDTVSLEDKVKINEFCHSSGIRFISSETRGLFGNTFVDLGDEFTVLDPTGEEPRTGMVSDIEPDGTVTMLDDNRHGLEDGNFVRFSEVEGLDKLNDGTLFKVEVLGPFAFRIGSVKEYGEYKKGGIFTEVKVPRKISFKSLKQQLSNPEFVFSDFAKFDRAAQLHLGFQALHQFAVRHNGELPRTMNDEDANELIKLVTDLSVQQPEVLGEGVDVNEDLIKELSYQARGDIPGVVAFFGGLVAQEVLKACSGKFTPLKQFMYFDSLESLPDPKNFPRNEKTTQPVNSRYDNQIAVFGLDFQKKIANSKVFLVGSGAIGCEMLKNWALLGLGSGSDGYIVVTDNDSIEKSNLNRQFLFRPKDVGKNKSEVAAEAVCAMNPDLKGKINAKIDKVGPETEEIFNDSFWESLDFVTNALDNVDARTYVDRRCVFYRKPLLESGTLGTKGNTQVIIPRLTESYSSSRDPPEKSIPLCTLRSFPNKIDHTIAWAKSLFQGYFTDSAENVNMYLTQPNFVEQTLKQSGDVKGVLESISDSLSSKPHNFEDCIKWARLEFEKKFNHDIKQLLFNFPKDAKTSNGEPFWSGAKRAPTPLEFDIYNNDHFHFVVAGASLRAYNYGIKSDDSNSKPNVDEYKSVIDHMIIPEFTPNANLKIQVNDDDPDPNANAANGSDEIDQLVSSLPDPSTLAGFKLEPVDFEKDDDTNHHIEFITACSNCRAQNYFIETADRQKTKFIAGRIIPAIATTTSLVTGLVNLELYKLIDNKTDIEQYKNGFVNLALPFFGFSEPIASPKGEYNNKKYDKIWDRFDIKGDIKLSDLIEHFEKDEGLEITMLSYGVSLLYASFFPPKKLKERLNLPITQLVKLVTKKDIPAHVSTMILEICADDKEGEDVEVPFITIHL

SEQ ID NO: 10 - protein, 147 aa, Artificial Sequence
  Ubch5a (Homo sapiens)
MALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAM

SEQ ID NO: 11 - protein, 154 aa, Artificial Sequence [sequence omitted]

SEQ ID NO: 12 - protein, 200 aa, Artificial Sequence
  E2-25K (Homo sapiens)
MANIAVQRIKREFKEVLKSEETSKNQIKVDLVDENFTELRGEIAGPPDTPYEGGRYQLEIKIPETYPFNPPKVRFITKIWHPNISSVTGAICLDILKDQWAAAMTLRTVLLSLQALLAAAEPDDPQDAVVANQYKQNPEMFKQTARLWAHVYAGAPVSSPEYTKKIENLCAMGFDRNAVIVALSSKSWDVETATELLLSN

SEQ ID NO: 13 - protein, 153 aa, Artificial Sequence
  Ubc13 (Saccharomyces cerevisiae)
MASLPKRIIKETEKLVSDPVPGITAEPHDDNLRYFQVTIEGPEQSPYEDGIFELELYLPDDYPMEAPKVRFLTKIYHPNIDRLGRICLDVLKTNWSPALQIRTVLLSIQALLASPNPNDPLANDVAEDWIKNEQGAKAKAREWTKLYAKKKPE

SEQ ID NO: 14 - protein, 137 aa, Artificial Sequence
  MMS2 (UEV - Ubiquitin-conjugating enzyme variant) (Saccharomyces cerevisiae)
MSKVPRNFRLLEELEKGEKGFGPESCSYGLADSDDITMTKWNGTILGPPHSNHENRIYSLSIDCGPNYPDSPPKVTFISKINLPCVNPTTGEVQTDFHTLRDWKRAYTMETLLLDLRKEMATPANKKLRQPKEGETF

SEQ ID NO: 15 - protein, 807 aa, Artificial Sequence
  E3 RSP5
MPSSISVKLVAAESLYKRDVFRSPDPFAVLTIDGYQTKSTSAAKKTLNPYWNETFKFDDINENSILTIQVFDQKKFKKKDQGFLGVVNVRVGDVLGHLDEDTATSSGRPREETITRDLKKSNDGMAVSGRLIVVLSKLPSSSPHSQAPSGHTASSSTNTSSTTRTNGHSTSSTRNHSTSHPSRGTAQAVESTLQSGTTAATNTATTSHRSTNSTSSATRQYSSFEDQYGRLPPGWERRTDNFGRTYYVDHNTRTTTWKRPTLDQTEAERGQLNANTELERRQHRGRTLPGGSSDNSSVTVQVGGGSNIPPVNGAAAAAFAATGGTTSGLGELPSGWEQRFTPEGRAYFVDHNTRTTTWVDPRRQQYIRTYGPTNTTIQQQPVSQLGPLPSGWEMRLTNTARVYFVDHNTKTTTWDDPRLPSSLDQNVPQYKRDFRRKVIYFRSQPALRILPGQCHIKVRRKNIFEDAYQEIMRQTPEDLKKRLMIKFDGEEGLDYGGVSREFFFLLSHEMFNPFYCLFEYSAYDNYTIQINPNSGINPEHLNYFKFIGRVVGLGVFHRRFLDAFFVGALYKMMLRKKVVLQDMEGVDAEVYNSLNWMLENSIDGVLDLTFSADDERFGEVVTVDLKPDGRNIEVTDGNKKEYVELYTQWRIVDRVQEQFKAFMDGFNEIPEDLVTVFDERELELLIGGIAEIDIEDWKKHTDYRGYQESDEVIQWFWKCVSEWDNEQRARLLQFTTGTSRIPVNGFKDLQGSDGPRRFTIEKAGEVQQLPKSHTCFNRVDLPQYVDYDSMKQKLTLAVEETIGFGQE

SEQ ID NO: 16 - protein, 1319 aa, Artificial Sequence
  E3 DOA10
MDVDSDVNVSRLRDELHKVANEETDTATFNDDAPSGATCRICRGEATEDNPLFHPCKCRGSIKYMHESCLLEWVASKNIDISKPGADVKCDICHYPIQFKTIYAENMPEKIPFSLLLSKSILTFFEKARLALTIGLAAVLYIIGVPLVWNMFGKLYTMMLDGSSPYPGDFLKSLIYGYDQSATPELTTRAIFYQLLQNHSFTSLQFIMIVILHIALYFQYDMIVREDVFSKMVFHKIGPRLSPKDLKSRLKERFPMMDDRMVEYLAREMRAHDENRQEQGHDRLNMPAAAADNNNNVINPRNDNVPPQDPNDHRNFENLRHVDELDHDEATEEHENNDSDNSLPSGDDSSRILPGSSSDNEEDEEAEGQQQQQQPEEEADYRDHIEPNPIDMWANRRAQNEFDDLIAAQQNAINRPNAPVFIPPPAQNRAGNVDQDEQDFGAAVGVPPAQANPDDQGQGPLVINLKLKLLNVIAYFIIAVVFTAIYLAISYLFPTFIGFGLLKIYFGIFKVILRGLCHLYYLSGAHIAYNGLTKLVPKVDVAMSWISDHLIHDIIYLYNGYTENTMKHSIFIRALPALTTYLTSVSIVCASSNLVSRGYGRENGMSNPTRRLIFQILFALKCTFKVFTLFFIELAGFPILAGVMLDFSLFCPILASNSRMLWVPSICAIWPPFSLFVYWTIGTLYMYWFAKYIGMIRKNIIRPGVLFFIRSPEDPNIKILHDSLIHPMSIQLSRLCLSMFIYAIFIVLGFGFHTRIFFPFMLKSNLLSVPEAYKPTSIISWKFNTILLTLYFTKRILESSSYVKPLLERYWKTIFKLCSRKLRLSSFILGKDTPTERGHIVYRNLFYKYIAAKNAEWSNQELFTKPKTLEQAEELFGQVRDVHAYFVPDGVLMRVPSSDIVSRNYVQTMFVPVTKDDKLLKPLDLERIKERNKRAAGEFGYLDEQNTEYDQYYIVYVPPDFRLRYMTLLGLVWLFASILMLGVTFISQALINFVCSFGFLPVVKLLLGERNKVYVAWKELSDISYSYLNIYYVCVGSVCLSKIAKDILHFTEGQNTLDEHAVDENEVEEVEHDIPERDINNAPVNNINNVEEGQGIFMAIFNSIFDSMLVKYNLMVFIAIMIAVIRTMVSWVVLTDGILACYNYLTIRVFGNSSYTIGNSKWFKYDESLLFVVWIISSMVNFGTGYKSLKLFFRNRNTSKLNFLKTMALELFKQGFLHMVIYVLPIIILSLVFLRDVSTKQIIDISHGSRSFTLSLNESFPTWTRMQDIYFGLLIALESFTFFFQATVLFIQWFKSTVQNVKDEVYTKGRALENLPDES

SEQ ID NO: 17 - protein, 278 aa, Artificial Sequence
  E3 MARCH5
MPDQALQQMLDRSCWVCFATDEDDRTAEWVRPCRCRGSTKWVHQACLQRWVDEKQRGNSTARVACPQCNAEYLIVFPKLGPVVYVLDLADRLISKACPFAAAGIMVGSIYWTAVTYGAVTVMQVVGHKEGLDVMERADPLFLLIGLPTIPVMLILGKMIRWEDYVLRLWRKYSNKLQILNSIFPGIGCPVPRIPAEANPLADHVSATRILCGALVFPTIATIVGKLMFSSVNSNLQRTILGGIAFVAIKGAFKVYFKQQQYLRQAHRKILNYPEQEEA

SEQ ID NO: 18 - protein, 1116 aa, Artificial Sequence
  His-SUMO-hGH-Ub(D)
METGLYHISHISHISHISHISHISSERASPGLNGLAALALYSPRASERTHRGLAASPLEAGLYASPLYSLYSGLAGLYGLATYRILELYSLEALYSVALILEGLYGLNASPSERSERGLAILEHISPHELYSVALLYSMETTHRTHRHISLEALYSLYSLEALYSGLASERTYRCYSGLNARGGLNGLYVALPRAMETASNSERLEAARGPHELEAPHEGLAGLYGLNARGILEALAASPASNHISTHRPRALYSGLALEAGLYMETGLAGLAGLAASPVALILEGLAVALTYRGLNGLAGLNTHRGLYGLYPHEPRATHRILEPRALEASERARGLEAPHEASPASNALAMETLEAARGALAHISARGLEAHISGLNLEAALAPHEASPTHRTYRGLNGLAPHEGLAGLAALATYRILEPRALYSGLAGLNLYSTYRSERPHELEAGLNASNPRAGLNTHRSERLEACYSPHESERGLASERILEPRATHRPRASERASNARGGLAGLATHRGLNGLNLYSSERASNLEAGLALEALEAARGILESERLEALEALEAILEGLNSERTRPLEAGLAPRAVALGLNPHELEAARGSERVALPHEALAASNSERLEAVALTYRGLYALASERASPSERASNVALTYRASPLEALEALYSASPLEAGLAGLAGLYILEGLNTHRLEAMETGLYARGLEAGLAASPGLYSERPRAARGTHRGLYGLNILEPHELYSGLNTHRTYRSERLYSPHEASPTHRASNSERHISASNASPASPALALEALEALYSASNTYRGLYLEALEATYRCYSPHEARGLYSASPMETASPLYSVALGLATHRPHELEAARGILEVALGLNCYSARGSERVALGLAGLYSERCYSGLYPHEMETGLNILEPHEVALARGTHRLEATHRGLYARGTHRILETHRLEAGLAVALGLAPRASERASPTHRILEGLAASNVALARGALAARGILEGLNASPARGGLAGLYILEPRAPRAASPGLNGLNARGLEAILEPHEALAGLYARGGLNLEAGLAASPGLYARGTHRLEASERASPTYRASNILEGLNARGGLASERTHRLEAHISLEAVALLEAARGLEAARGGLYGLYASP

SEQ ID NO: 19 - protein, 1116 aa, Artificial Sequence
  His-SUMO- hGH-Ub(A)
METGLYHISHISHISHISHISHISSERASPGLNGLAALALYSPRASERTHRGLAASPLEAGLYASPLYSLYSGLAGLYGLATYRILELYSLEALYSVALILEGLYGLNASPSERSERGLAILEHISPHELYSVALLYSMETTHRTHRHISLEALYSLYSLEALYSGLASERTYRCYSGLNARGGLNGLYVALPRAMETASNSERLEAARGPHELEAPHEGLAGLYGLNARGILEALAASPASNHISTHRPRALYSGLALEAGLYMETGLAGLAGLAASPVALILEGLAVALTYRGLNGLAGLNTHRGLYGLYPHEPRATHRILEPRALEASERARGLEAPHEASPASNALAMETLEAARGALAHISARGLEAHISGLNLEAALAPHEASPTHRTYRGLNGLAPHEGLAGLAALATYRILEPRALYSGLAGLNLYSTYRSERPHELEAGLNASNPRAGLNTHRSERLEACYSPHESERGLASERILEPRATHRPRASERASNARGGLAGLATHRGLNGLNLYSSERASNLEAGLALEALEAARGILESERLEALEALEAILEGLNSERTRPLEAGLAPRAVALGLNPHELEAARGSERVALPHEALAASNSERLEAVALTYRGLYALASERASPSERASNVALTYRASPLEALEALYSASPLEAGLAGLAGLYILEGLNTHRLEAMETGLYARGLEAGLAASPGLYSERPRAARGTHRGLYGLNILEPHELYSGLNTHRTYRSERLYSPHEASPTHRASNSERHISASNASPASPALALEALEALYSASNTYRGLYLEALEATYRCYSPHEARGLYSASPMETASPLYSVALGLATHRPHELEAARGILEVALGLNCYSARGSERVALGLAGLYSERCYSGLYPHEMETGLNILEPHEVALARGTHRLEATHRGLYARGTHRILETHRLEAGLAVALGLAPRASERASPTHRILEGLAASNVALARGALAARGILEGLNASPARGGLAGLYILEPRAPRAASPGLNGLNARGLEAILEPHEALAGLYARGGLNLEAGLAASPGLYARGTHRLEASERASPTYRASNILEGLNLYSGLASERTHRLEAHISLEAVALLEAARGPRAARGGLYGLYASP

SEQ ID NO: 20 - protein, 804 aa, Artificial Sequence
  Ub(A)-hGH
METGLNILEPHEVALARGTHRLEATHRGLYARGTHRILETHRLEAGLAVALGLAPRASERASPTHRILEGLAASNVALARGALAARGILEGLNASPARGGLAGLYILEPRAPRAASPGLNGLNARGLEAILEPHEALAGLYARGGLNLEAGLAASPGLYARGTHRLEASERASPTYRASNILEGLNLYSGLASERTHRLEAHISLEAVALLEAARGPRAARGGLYGLYASPPHEPRATHRILEPRALEASERARGLEAPHEASPASNALAMETLEAARGALAHISARGLEAHISGLNLEAALAPHEASPTHRTYRGLNGLAPHEGLAGLAALATYRILEPRALYSGLAGLNLYSTYRSERPHELEAGLNASNPRAGLNTHRSERLEACYSPHESERGLASERILEPRATHRPRASERASNARGGLAGLATHRGLNGLNLYSSERASNLEAGLALEALEAARGILESERLEALEALEAILEGLNSERTRPLEAGLAPRAVALGLNPHELEAARGSERVALPHEALAASNSERLEAVALTYRGLYALASERASPSERASNVALTYRASPLEALEALYSASPLEAGLAGLAGLYILEGLNTHRLEAMETGLYARGLEAGLAASPGLYSERPRAARGTHRGLYGLNILEPHELYSGLNTHRTYRSERLYSPHEASPTHRASNSERHISASNASPASPALALEALEALYSASNTYRGLYLEALEATYRCYSPHEARGLYSASPMETASPLYSVALGLATHRPHELEAARGILEVALGLNCYSARGSERVALGLAGLYSERCYSGLYPHE

SEQ ID NO: 21 - protein, 1116 aa, Artificial Sequence
  SUMO-Ub(A)-hGH
METGLYHISHISHISHISHISHISSERASPGLNGLAALALYSPRASERTHRGLAASPLEAGLYASPLYSLYSGLAGLYGLATYRILELYSLEALYSVALILEGLYGLNASPSERSERGLAILEHISPHELYSVALLYSMETTHRTHRHISLEALYSLYSLEALYSGLASERTYRCYSGLNARGGLNGLYVALPRAMETASNSERLEAARGPHELEAPHEGLAGLYGLNARGILEALAASPASNHISTHRPRALYSGLALEAGLYMETGLAGLAGLAASPVALILEGLAVALTYRGLNGLAGLNTHRGLYGLYMETGLNILEPHEVALARGTHRLEATHRGLYARGTHRILETHRLEAGLAVALGLAPRASERASPTHRILEGLAASNVALARGALAARGILEGLNASPARGGLAGLYILEPRAPRAASPGLNGLNARGLEAILEPHEALAGLYLYSGLNLEAGLAASPGLYARGTHRLEASERASPTYRASNILEGLNARGGLASERTHRLEAHISLEAVALLEAARGPRAARGGLYGLYASPPHEPRATHRILEPRALEASERARGLEAPHEASPASNALAMETLEAARGALAHISARGLEAHISGLNLEAALAPHEASPTHRTYRGLNGLAPHEGLAGLAALATYRILEPRALYSGLAGLNLYSTYRSERPHELEAGLNASNPRAGLNTHRSERLEACYSPHESERGLASERILEPRATHRPRASERASNARGGLAGLATHRGLNGLNLYSSERASNLEAGLALEALEAARGILESERLEALEALEAILEGLNSERTRPLEAGLAPRAVALGLNPHELEAARGSERVALPHEALAASNSERLEAVALTYRGLYALASERASPSERASNVALTYRASPLEALEALYSASPLEAGLAGLAGLYILEGLNTHRLEAMETGLYARGLEAGLAASPGLYSERPRAARGTHRGLYGLNILEPHELYSGLNTHRTYRSERLYSPHEASPTHRASNSERHISASNASPASPALALEALEALYSASNTYRGLYLEALEATYRCYSPHEARGLYSASPMETASPLYSVALGLATHRPHELEAARGILEVALGLNCYSARGSERVALGLAGLYSERCYSGLYPHE

SEQ ID NO: 22 - DNA, 60 bp, Artificial Sequence
  Signal peptide (IgGk)
atggaaactgatactctgctgctgtgggtgctgctgctgtgggtgcccggctcaactggt

SEQ ID NO: 23 - DNA, 231 bp, Artificial Sequence
  Ub (A)
atgcagatcttcgtgaggaccctgacagatcggaccatcacactggaggtggagccaagcgacaccatcgagaacgtgagggccagaatccaggaccgggagggcatcccccctgatcagcagagactgatcttcgctggccgccagctggaggacggaaggaccctgagcgattacaatatccagaaagagtctacactgcacctggtgctgagaccgcgcgtcgtggat

SEQ ID NO: 24 - DNA, 36 bp, Artificial Sequence
  Hinge (IgG1)
gagccaaaatcttgtgacaaaactcatacatgtccc

SEQ ID NO: 25 - DNA, 660 bp, Artificial Sequence
  Fc (IgG1)
ccatgtcccgcacctgaactgctgggcggacctagcgtgtttctgttcccacctaagccaaaggacaccctgatgatctccaggacccccgaggtgacatgcgtggtggtggacgtgagccacgaggaccccgaggtgaagttcaactggtacgtggatggcgtggaggtgcataatgccaagacaaagccaagggaggagcagtacaacagcacctatcgggtggtgtctgtgctgacagtgctgcaccaggactggctgaacggcaaggagtataagtgcaaggtgtctaataaggccctgcccgctcctatcgagaagaccatctccaaggccaagggccagccaagagagccccaggtgtacacactgccccctagccgcgacgagctgaccaagaaccaggtgtctctgacatgtctggtgaagggcttctatccttctgatatcgctgtggagtgggagtccaatggccagccagagaacaattacaagaccacaccacccgtgctggactctgatggctccttctttctgtattccaagctgaccgtggataagagcagatggcagcagggcaacgtgttctcctgtagcgtgatgcatgaagcactgcataatcactatacccagaagtcactgtcactgagtcccggtaaa

SEQ ID NO: 26 - protein, 20 aa, Artificial Sequence
  Signal peptide (IgGk)
METDTLLLWVLLLWVPGSTG

SEQ ID NO: 27 - protein, 77 aa, Artificial Sequence
  Ub(A)
MQIFVRTLTDRTITLEVEPSDTIENVRARIQDREGIPPDQQRLIFAGRQLEDGRTLSDYNIQKESTLHLVLRPRVVD

SEQ ID NO: 28 - protein, 12 aa, Artificial Sequence
  Hinge (IgG1)
EPKSCDKTHTCP

SEQ ID NO: 29 - protein, 660 aa, Artificial Sequence
  Fc (IgG1)
PRACYSPRAALAPRAGLALEALEAGLYGLYPRASERVALPHELEAPHEPRAPRALYSPRALYSASPTHRLEAMETILESERARGTHRPRAGLAVALTHRCYSVALVALVALASPVALSERHISGLAASPPRAGLAVALLYSPHEASNTRPTYRVALASPGLYVALGLAVALHISASNALALYSTHRLYSPRAARGGLAGLAGLNTYRASNSERTHRTYRARGVALVALSERVALLEATHRVALLEAHISGLNASPTRPLEAASNGLYLYSGLATYRLYSCYSLYSVALSERASNLYSALALEAPRAALAPRAILEGLALYSTHRILESERLYSALALYSGLYGLNPRAARGGLAPRAGLNVALTYRTHRLEAPRAPRASERARGASPGLALEATHRLYSASNGLNVALSERLEATHRCYSLEAVALLYSGLYPHETYRPRASERASPILEALAVALGLATRPGLASERASNGLYGLNPRAGLAASNASNTYRLYSTHRTHRPRAPRAVALLEAASPSERASPGLYSERPHEPHELEATYRSERLYSLEATHRVALASPLYSSERARGTRPGLNGLNGLYASNVALPHESERCYSSERVALMETHISGLAALALEAHISASNHISTYRTHRGLNLYSSERLEASERLEASERPRAGLYLYS

SEQ ID NO: 30 - protein, 5 aa, Artificial Sequence
  Linker
  REPEAT (1)..(5): The entire sequence of amino acids 1-5 can be repeated one to thirty times.
GGGGS

SEQ ID NO: 31 - protein, 5 aa, Artificial Sequence
  Linker
  REPEAT (1)..(5): The entire sequence of amino acids 1-5 can be repeated one to thirty times.
EAAAK

SEQ ID NO: 32 - protein, 220 aa, Artificial Sequence
  Fc (IgG1)
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK